(19) United States
(12) Patent Application Publication
(10) Pub. No.: US 2006/0128710 A1
(43) Pub. Date: Jun. 15, 2006

(54) Antagonists to the vanilloid receptor subtype 1 (VR1) and uses thereof

(76) Inventors: Chih-Hung Lee, Vernon Hills, IL (US); Brian S. Brown, Evanston, IL (US); Ryan G. Keddy, Beach Park, IL (US); Richard J. Perner, Gurnee, IL (US); John R. Koenig, Chicago, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/292,979

(22) Filed: Dec. 2, 2005

Related U.S. Application Data
(60) Provisional application No. 60/634,612, filed on Dec. 9, 2004.

(51) Int. Cl. A61K 31/519; A61K 31/503; A61K 31/4745; C07D 487/02; C07D 471/02

(52) U.S. Cl. 514/248; 514/264.11; 514/265.1; 514/300; 544/236; 544/279; 544/280; 546/113; 546/122

(57) ABSTRACT

Compounds having formula (I) or formula (II) 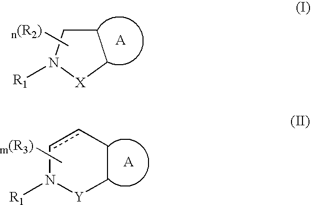
or a pharmaceutically acceptable salt, prodrug, or salt of a prodrug thereof, wherein A, N, X, Y, R₁, R₂ and R₃ are as defined in the specification. These compounds are particularly useful in the treatment of pain, inflammatory hyperalgesia, and urinary dysfunctions, such as bladder overactivity and urinary incontinence.

This application claims priority to U.S. Provisional Application Ser. No. 60/634,612 filed on Dec. 9, 2004.

### TECHNICAL BACKGROUND

The present invention relates to compounds of formula (I) or formula (II), which are useful for treating disorders caused by or exacerbated by vanilloid receptor activity and pharmaceutical compositions containing compounds of formula (I) or formula (II). The compounds of the present invention are useful in treating pain, inflammatory hyperalgesia, and urinary dysfunctions, such as bladder overactivity and urinary incontinence.

### BACKGROUND OF THE INVENTION

Nociceptors are primary sensory afferent (C and Aδ fibers) neurons that are activated by a wide variety of noxious stimuli including chemical, mechanical, thermal, and proton (pH<6) modalities. The lipophillic vanilloid, capsaicin, activates primary sensory fibers via a specific cell surface capsaicin receptor, cloned as VR1. The intradermal administration of capsaicin is characterized by an initial burning or hot sensation followed by a prolonged period of analgesia. The analgesic component of VR1 receptor activation is thought to be mediated by a capsaicin-induced desensitization of the primary sensory afferent terminal. Thus, the long lasting anti-nociceptive effects of capsaicin have prompted the clinical use of capsaicin analogs as analgesic agents (Nolano et al., Pain, Vol 81, pages 135-145, 1999). Further, capsazepine, a capsaicin receptor antagonist can reduce inflammation-induced hyperalgesia in animal models. VR1 receptors are also localized on sensory afferents, which innervate the bladder. Capsaicin or resiniferatoxin has been shown to ameliorate incontinence symptoms upon injection into the bladder (Fowler, Urology, Vol. 55, pages 60-64, 2000).

The VR1 receptor has been called a “polymodal detector” of noxious stimuli since it can be activated in several ways. The receptor channel is activated by capsaicin and other vanilloids and thus is classified as a ligand-gated ion channel. VR1 receptor activation by capsaicin can be blocked by the competitive VR1 receptor antagonist, capsazepine. The channel can also be activated by protons. Under mildly acidic conditions (pH 6-7), the affinity of capsaicin for the receptor is increased, whereas at pH<6, direct activation of the channel occurs. In addition, when membrane temperature reaches 43° C., the channel is opened. Thus heat can directly gate the channel in the absence of ligand. The capsaicin analog, capsazepine, which is a competitive antagonist of capsaicin, blocks activation of the channel in response to capsaicin, acid, or heat (Caterina et al., Nature, Vol 389, pages 816-824).

The channel is a nonspecific cation conductor. Both extracellular sodium and calcium enter through the channel pore, resulting in cell membrane depolarization. This depolarization increases neuronal excitability, leading to action potential firing and transmission of a noxious nerve impulse to the spinal cord. In addition, depolarization of the peripheral terminal can lead to release of inflammatory peptides such as, but not limited to, substance P and CGRP, leading to enhanced peripheral sensitization of tissue.

Recently, two groups have reported the generation of a “knock-out” mouse lacking the VR1 receptor (VR1 (-/-)). Electrophysiological studies of sensory neurons (dorsal root ganglia) from these animals revealed a marked absence of responses evoked by noxious stimuli including capsaicin, heat, and reduced pH. These animals did not display any overt signs of behavioral impairment and showed no differences in responses to acute non-noxious thermal and mechanical stimulation relative to wild-type mice. The VR1 (-/-) mice also did not show reduced sensitivity to nerve injury-induced mechanical or thermal nociception. However, the VR1 knock-out mice were insensitive to the noxious effects of intradermal capsaicin, exposure to intense heat (50-55° C.), and failed to develop thermal hyperalgesia following the intradermal administration of carrageenan (Caterina et al., Science, Vol. 288, pages 306-313, 2000; Davis et al, Nature, Vol. 405, pages 183-187, 2000).

The compounds of the present invention are novel VR1 antagonists and have utility in treating pain, inflammatory hyperalgesia, and urinary dysifunctions, such as bladder overactivity and urinary incontinence.

### SUMMARY OF THE PRESENT INVENTION

The present invention discloses novel compounds, a method for inhibiting the VR1 receptor in mammals using these compounds, pharmaceutical compositions including these compounds, and methods of treating a disorder wherein the disorder is ameliorated by inhibiting vanilloid receptor subtype 1 (VR1) receptor in a host mammal in need of such treatment comprising administering a therapeutically effective amount of a compound of formula (I) and formula (II) as defined in claim 1 or a pharmaceutically acceptable salt thereof, and wherein the disorder is selected form the group consisting of pain, inflammatory hyperalgesia, bladder overactivity and urinary incontinence.

More particularly, the present invention is directed to compounds of formula (I) or formula (II) 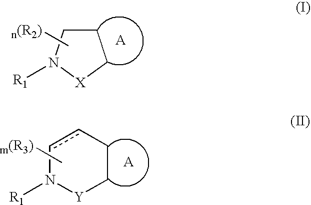
or a pharmaceutically acceptable salt, prodrug, or salt of a prodrug thereof, wherein

X is CH₂ or C(O);

Y is CH₂ or C(O);

R₁ is hydrogen, —C(O)R_{c}, —C(O)NR_{c}R_{d}, —S(O)₂R_{c}, aryl, heteroaryl, heterocycle, cycloalkyl or cycloalkenyl; wherein each R₁ is substituted with 0, 1, 2, 3 or 4 substituents selected from the group consisting of halo, —CN, —NO₂, alkyl, alkenyl, alkynyl, haloalkyl, haloalkoxy, —OR_{d}, —NR_{d}Rₑ, —SR_{d}, —S(O)R_{d}, —S(O)₂R_{d}, —alkylSR_{d}, —alkylS(O)R_{d}, —alkylS(O)₂R_{d}, —alkylOR_{d}, and —alkylNR_{d}Rₑ;

R₂ is halo, —CN, —NO₂, alkyl, alkenyl, alkynyl, haloalkyl, haloalkoxy, —OH, —O(alkyl), —NH_{2,} —N(alkyl)_{2,} —NR_{d}Rₑ, or —N(H)alkyl;

R₃ is halo, —CN, —NO₂, alkyl, alkenyl, alkynyl, haloalkyl, haloalkoxy, —OH, —O(alkyl), —NH_{2,} —N(alkyl)₂, or —N(H)alkyl;

[symbol] is a single bond or a double bond;

m is 0, 1, 2 or 3;

n is 0, 1 or 2;

A is

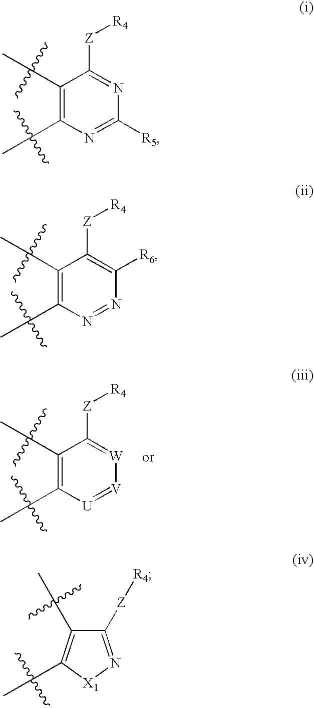

Z is NH, O, or S;

R₄ is aryl, heteroaryl, heterocycle, cycloalkyl or cycloalkenyl; wherein each R₄ is substituted with 0, 1, 2, 3 or 4 substituents selected from the group consisting of halo, —CN, —NO₂, alkyl, alkenyl, alkynyl, haloalkyl, haloalkoxy, —OR_{d}, —OC(O)R_{d}, —NR_{d}Rₑ, —N(Rₑ)C(O)NR_{d}Rₑ, —N(Rₑ)C(O)OR_{d}, —N(Rₑ)C(O)NR_{d}Rₑ, —N(Rₑ)S(O)₂R_{d}, —N(Rₑ)S(O)₂NR_{d}Rₑ, —SR_{d}, —S(O)R_{d}, —S(O)₂R_{d}, —S(O)₂NR_{d}Rₑ, —C(O)OR_{d}, —C(O)NR_{d}Rₑ, heterocycle, —alkylOR_{d}, —alkylOC(O)R_{d}, —alkylNR_{d}Rₑ, —alkylN(Rₑ)C(O)NR_{d}Rₑ, —alkylN(Rₑ)C(O)OR_{d}, alkylN(Rₑ)C(O)NR_{d}Rₑ, —alkylN(Rₑ)S(O)₂R_{d}, —alkylN(Rₑ)S(O)₂NR_{d}Rₑ, —alkylSR_{d}, —alkylS(O)R_{d}, —alkylS(O)₂R_{d}, —alkylS(O)₂NR_{d}Rₑ, —alkylC(O)OR_{d}, and —alkylC(O)NR_{d}Rₑ;

R₅ is H, halo, haloalkyl, haloalkoxy, —CN, —NO₂, alkyl, —ORₐ, —SRₐ, —S(O)Rₐ, —SO₂Rₐ, —alkylNₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₆ is H, halo, haloalkyl, haloalkoxy, —CN, —NO_{2,} alkyl, —OR, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

U is CR₇ or N;

V is CR₈ or N;

W is CR₉ or N;

provided that only one of U, V and W is N;

R₇ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₈ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₉ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

X₁ is N, O, SO₂, or S;

Rₐ is hydrogen, alkyl, aryl or arylalkyl;

R_{b} is hydrogen, alkyl, aryl or arylalkyl;

alternatively, Rₐ and R_{b}together with the nitrogen atom they are attached to, form a 4, 5 or 6 membered ring selected from the group consisting of heterocycle or heteroaryl, wherein each ring is substituted with 0, 1, 2, 3 or 4 susbstituents selected from the group consisting of oxo, alkyl, —OR_{d}, —NR_{d}Rₑ, —SR_{d}, —S(O)R_{d}, —S(O)₂R_{d}, —alkylOR_{d}, —alkylNR_{d}Rₑ, —alkylSR_{d}, —alkylS(O)R_{d}, —alkylS(O)₂R_{d}, —CN, —NO₂, halo, haloalkyl, and haloalkoxy;

R_{c} is aryl or heteroaryl; wherein each R_{c} is substituted with 0, 1, 2, 3, 4, or 5 substituents selected from the group consisting of alkyl, alkenyl, alkynyl, —OR_{d}, —NR_{d}Rₑ, —SR_{d}, —S(O)R_{d}, —S(O)₂R_{d}, —alkylOR_{d}, —alkylNR_{d}Rₑ, —alkylSR_{d}, —alkylS(O)R_{d}, —alkylS(O)₂R_{d}, —CN, —NO₂, halo, haloalkyl, and haloalkoxy;

R_{d} is hydrogen, alkyl, aryl or arylalkyl; and

Rₑ is hydrogen, alkyl, aryl or arylalkyl.

### DETAILED DESCRIPTION OF THE PRESENT INVENTION

### (1) EMBODIMENTS

The present invention discloses a compound of formula (I) 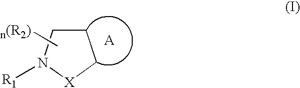
or a pharmaceutically acceptable salt or prodrug thereof, wherein
or a pharmaceutically acceptable salt, prodrug, or salt of a prodrug thereof, wherein

X is CH₂ or C(O);

R₁ is —C(O)R_{c}, —C(O)NR_{c}R_{d}, —S(O)₂R_{c}, aryl, heteroaryl, heterocycle, cycloalkyl or cycloalkenyl; wherein each R₁ is substituted with 0, 1, 2, 3 or 4 substituents selected from the group consisting of halo, —CN, —NO₂, alkyl, alkenyl, alkynyl, haloalkyl, haloalkoxy, —OR_{d}, —NR_{d}Rₑ, —SR_{d}, —S(O)R_{d}, —S(O)₂R_{d}, —alkylSR_{d}, —alkylS(O)R_{d}, —alkylS(O)₂R_{d}, —alkylOR_{d}, and —alkylNR_{d}Rₑ;

R₂ is halo, —CN, —NO₂, alkyl, alkenyl, alkynyl, haloalkyl, haloalkoxy, —OH, —O(alkyl), —NH_{2,} —N(alkyl)₂, —NR_{d}, or, —N(H)alkyl;

n is 0, 1 or 2;

A is

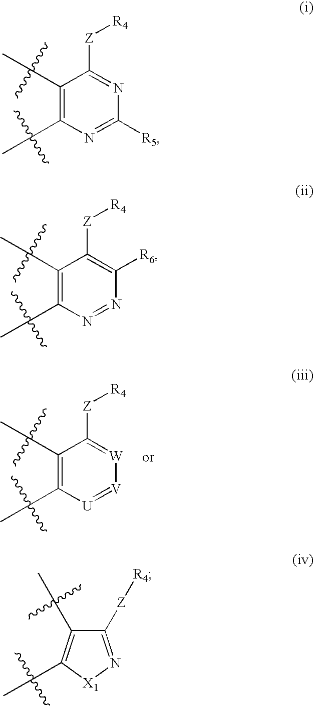

Z is NH, O, or S;

R₄ is aryl, heteroaryl, heterocycle, cycloalkyl or cycloalkenyl; wherein each R₄ is substituted with 0, 1, 2, 3 or 4 substituents selected from the group consisting of halo, —CN, —NO₂, alkyl, alkenyl, alkynyl, haloalkyl, haloalkoxy, —OR_{d}, —OC(O)R_{d}, —NR_{d}Rₑ, —N(Rₑ)C(O)NR_{d}Rₑ, —N(Rₑ)C(O)OR_{d}, —N(Rₑ)C(O)NR_{d}Rₑ, —N(Rₑ)S(O)₂R_{d}, —N(Rₑ)S(O)₂NR_{d}, —SR_{d}, —S(O)R_{d}, —S(O)₂R_{d}, —S(O)₂NR_{d}Rₑ, —C(O)OR_{d}, —C(O)NR_{d}Rₑ, heterocycle, —alkylOR_{d}, —alkylOC(O)R_{d}, —alkylNR_{d}Rₑ, —alkylN(Rₑ)C(O)NR_{d}Rₑ, —alkylN(Rₑ)C(O)OR_{d}, —alkylN(Rₑ)C(O)NR_{d}Rₑ, —alkylN(Rₑ)S(O)₂R_{d}, —alkylN(Rₑ)S(O)₂NR_{d}Rₑ, —alkylSR_{d}, —alkylS(O)R_{d}, —alkylS(O)₂R_{d}, —alkylS(O)₂NR_{d}Rₑ, —alkylC(O)OR_{d}, and —alkylC(O)NR_{d}Rₑ;

R₅ is H, halo, haloalkyl, haloalkoxy, —CN, —NO₂, alkyl, —ORₐ, —SRₐ, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₆ is H, halo, haloalkyl, haloalkoxy, —CN, —NO₂, alkyl, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

U is CR₇ or N;

V is CR₈ or N;

W is CR₉ or N;

provided that only one of U, V and W is N;

R₇ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₈ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₉ is H, alkyl, halo, haloalkyl, —CN, —NO₂, —ORₐ, —SRₐ, —NRₐR_{b}, —S(O)Rₐ, SO₂Rₐ, —alkylNRₐR_{b}, —alkylORₐ, —alkylSFₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

X₁ is N, O, SO₂, or S;

R_{c} is hydrogen, alkyl, aryl or arylalkyl;

R_{b} is hydrogen, alkyl, aryl or arylalkyl;

alternatively, Rₐ and R_{b}, together with the nitrogen atom they are attached to, form a 4, 5 or 6 membered ring selected from the group consisting of heterocycle or heteroaryl, wherein each ring is substituted with 0, 1, 2, 3 or 4 susbstituents selected from the group consisting of oxo, alkyl, —OR_{d}, —NR_{d}Rₑ, —SR_{d}, —S(O)R_{d}, —S(O)₂R_{d}, —alkylOR_{d}, —alkylNR_{d}Rₑ, —alkylSR_{d}, —alkylS(O)R_{d}, —alkylS(O)₂R_{d}, —CN, —NO₂, halo, haloalkyl, and haloalkoxy;

R_{c} is aryl or heteroaryl; wherein each Rₑ is substituted with 0, 1, 2, 3, 4, or 5 substituents selected from the group consisting of alkyl, alkenyl, alkynyl, —OR_{d}, —NR_{d}Rₑ, —SR_{d}, —S(O)R_{d}, —S(O)₂R_{d}, —alkylOR_{d}, —alkylNR_{d}Rₑ, —alkylSR_{d}, —alkylS(O)R_{d}, —alkylS(O)₂R_{d}, —CN, —NO₂, halo, haloalkyl, and haloalkoxy;

R_{d} is hydrogen, alkyl, aryl or arylalkyl; and

Rₑ is hydrogen, alkyl, aryl or arylalkyl.

Preferred compounds are those in which X is CH₂ or C(O);

A is 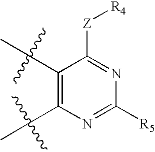
and n, R₁, R₂, Z, R₄ and R₅ are as defined above. Other preferred compounds are those wherein X is CH₂ or C(O);

A is 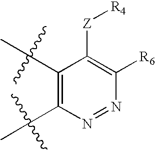
and n, R₁, R₂, Z, R₄ and R₆ are as defined above. Also preferred compounds include those wherein X is CH₂ or C(O);

A is 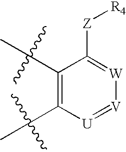
and n, R₁, R₂, U, V, W, Z and R₄ are as defined above. Most preferred us a compound wherein X is CH₂; U is N; V is CR_{8;} W is CR₉; and Z is as defined above. The present invention also includes preferred compounds in which X is CH₂; U is CR₇; V is N; W is CR₉; and Z is as defined above, or wherein X is CH₂; U is CR₇; V is CR₈; W is N; and Z is as defined above. Other preferred compounds include those wherein X is C(O); U is N; V is CR₈; W is CR₉; and Z is as defined above. Also included are compounds wherein X is C(O); U is CR₇; V is N; W is CR₉; and Z is as defined above; or wherein X is C(O); U is CR₇; V is CR_{8;} W is N; and Z is as defined above.

The present invention also includes compounds of formula (I) wherein X is CH₂ or C(O); A is 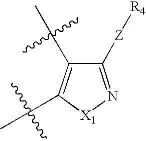
and n, R₁, R₂, X₁, Z and R₄ are as defined above. Preferably X is CH₂; Z is NH; and X₁ is N(R_{d}), O or S; or X is CH₂; Z is O; and X₁ is N(R_{d}), O or S. Other preferred compounds include compound wherein X is CH₂; Z is NH; and X₁ is N(R_{d}), O or S; or wherein X is C(O); Z is NH; and X₁ is N(R_{d}), O or S; or wherein X is C(O); Z is O; and X₁ is N(R_{d}), O or S; or also wherein X is C(O); Z is NH; and X₁ is N(R_{d}), O or S.

In another embodiment, the present invention claims a compound of formula (II) 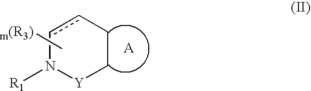
or a pharmaceutically acceptable salt, prodrug, or salt of a prodrug thereof, wherein

Y is CH₂ or C(O);

R₁ is —C(O)R, —C(O)NR_{c}R_{d}, —S(O)₂R_{c}, aryl, heteroaryl, heterocycle, cycloalkyl or cycloalkenyl; wherein each R₁ is substituted with 0, 1, 2, 3 or 4 substituents selected from the group consisting of halo, —CN, —NO₂, alkyl, alkenyl, alkynyl, haloalkyl, haloalkoxy, —OR_{d}, —NR_{d}R_{d}, —SR_{d}, —S(O)R_{d}, —S(O)₂R_{d}, —alkylSR_{d}, —alkylS(O)R_{d}, —alkylS(O)₂R_{d}, —alkylOR_{d}, and —alkylNR_{d}Rₑ;

R₃ is halo, —CN, —NO₂, alkyl, alkenyl, alkynyl, haloalkyl, haloalkoxy, —OH, —O(alkyl), —NH_{2,} —N(alkyl)_{2,} or —N(H)alkyl;

[symbol] is a single bond or a double bond;

m is 0, 1, 2 or 3;

A is

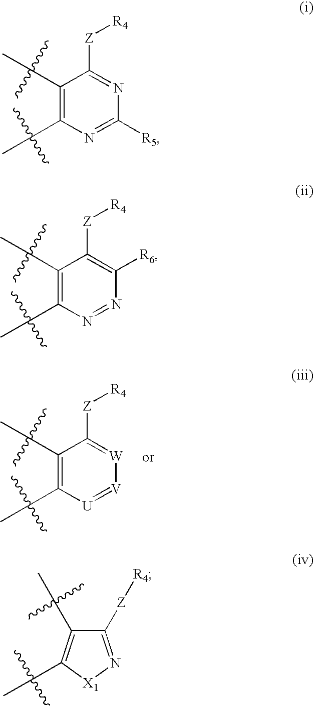

Z is NH, O, or S;

R₄ is aryl, heteroaryl, heterocycle, cycloalkyl or cycloalkenyl; wherein each R₄ is substituted with 0, 1, 2, 3 or 4 substituents selected from the group consisting of halo, —CN, —NO₂, alkyl, alkenyl, alkynyl, haloalkyl, haloalkoxy, —OR_{d}, —OC(O)R_{d}, —NR_{d}Rₑ, —N(Rₑ)C(O)NR_{d}Rₑ, —N(Rₑ)C(O)OR_{d}, —N(Rₑ)C(O)NR_{d}, —N(Rₑ)S(O)₂R_{d}, —N(Rₑ)S(O)₂NRₑ, —SR_{d}, —S(O)R_{d}, —S(O)₂R_{d}, —S(O)₂NR_{d}Rₑ, —C(O)OR_{d}, —C(O)NR_{d}Rₑ, heterocycle, —alkylOR_{d}, —alkylOC(O)R_{d}, —alkylNR_{d}Rₑ, —alkylN(Rₑ)C(O)NR_{d}R_{c}, —alkylN(Rₑ)C(O)OR_{d}, —alkylN(Rₑ)C(O)NR_{d}Rₑ, —alkylN(Rₑ)S(O)₂R_{d}, —alkylN(Rₑ)S(O)₂NR_{d}Rₑ, —alkylSR_{d}, —alkylS(O)R_{d}, —alkylS(O)₂R_{d}, —alkylS(O)₂NR_{d}Rₑ, —alkylC(O)OR_{d}, and —alkylC(O)NR_{d}Rₑ;

R₅ is H, halo, haloalkyl, haloalkoxy, —CN, —NO₂, alkyl, —ORₐ, —SRₐ, —S(O)Rₐ, —SO₂Rₐ, —alkylNR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₆ is H, halo, haloalkyl, haloalkoxy, —CN, —NO₂, alkyl, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

U is CR₇ or N;

V is CR₈ or N;

W is CR₉ or N;

provided that only one of U, V and W is N;

R₇ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

R₈ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)ₐ, —alkylS(O)₂Rₐ, —OC(O)Rₐ, —C(O)ORₐ, —C(O)Rₐ, —C(O)NRₐR_{b}, or R_{c};

R₉is H, alkyl, halo, haloalkyl, —CN, —NO₂, —ORₐ, —SRₐ, NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};

X₁ is N, O, SO₂, or S;

Rₐ is hydrogen, alkyl, aryl or arylalkyl;

R_{b} is hydrogen, alkyl, aryl or arylalkyl;

alternatively, Rₐ and R_{b}, together with the nitrogen atom they are attached to, form a 4, 5 or 6 membered ring selected from the group consisting of heterocycle or heteroaryl, wherein each ring is substituted with 0, 1, 2, 3 or 4 susbstituents selected from the group consisting of oxo, alkyl, —O, —NR_{d}, —SR_{d}, —S(O)R_{d}, —S(O)₂R_{d}, —alkylOR_{d}, —alkylNR_{d}Rₑ, —alkylSR_{d}, —alkylS(O)R_{d}, —alkylS(O)₂R_{d}, —CN, —NO₂, halo, haloalkyl, and haloalkoxy;

R_{c} is aryl or heteroaryl; wherein each R_{c} is substituted with 0, 1, 2, 3, 4, or 5 substituents selected from the group consisting of alkyl, alkenyl, alkynyl, —OR_{d}, —NR_{R}ₑ, —SR_{d}, —S(O)R_{d}, —S(O)₂R_{d}, —alkylOR_{d}, —alkylNR_{d}, —alkylSR_{d}, —alkylS(O)R_{d}, —alkylS(O)₂R_{d}, —CN, —NO₂, halo, haloalkyl, and haloalkoxy;

R_{d} is hydrogen, alkyl, aryl or arylalkyl; and

Rₑ is hydrogen, alkyl, aryl or arylalkyl.

Preferred compounds are those wherein Y is CH₂ or C(O); A is 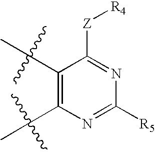
and m, R₁, R₃, Z, R₄ and R₅ are as defined above; or wherein Y is CH₂; Z is NH; and m, R₁, R₃, R₄ and R₅ are as defined above. Preferred compounds include those in which R₁ is arylalkyl and R₄ is aryl, those in which R₁ is heteroaryl and R₄ is aryl; and those in which R₁ is hydrogen and R₄ is aryl. Other preferred compounds include those of formula (II) in which Y is CH₂ and Z is O or S; and those in which Y is C(O) and Z is NH, O or S.

The present invention also includes compound of formula (II) wherein Y is CH₂ or C(O); A is 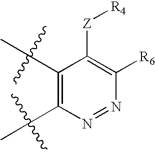
and m, R₁, R₃, Z, R₄ and R₅ are as defined above. Preferredcompounds include those in which Y is CH₂ and Z is NH, O or S. Also included are those compounds in which Y is C(O) and Z is NH, O or S.

Other compounds of the present invention are those compounds of formula (II) wherein Y is CH₂ or C(O); A is 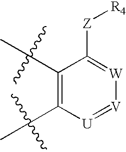
and m, R₁, R₃, U, V, W, Z and R₄ are as defined above. Preferably wherein Y is CH₂; U is N; V is CR_{8;} and W is CR₉; or wherein Y is CH₂; U is CR₇; V is N; and W is CR₉; or wherein Y is CH₂; U is CR₇; V is CR_{8;} and W is N. Other preferred compounds include those in which Y is C(O); U is N; V is CR₈; and W is CR₉; or those in which Y is C(O); U is CR₇; V is N; and W is CR₉; or those compounds in which Y is C(O); U is CR₇; V is CR_{8;} and W is N.

Other compounds included in the present invention are those compounds of formula (II), wherein Y is CH₂ or C(O);

A is 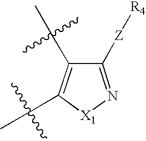
and m, R₁, R₃, X₁, Z and R₄ are as defined above. Preferred compounds include those wherein Y is CH₂; Z is NH; and X₁ is N(R_{d}), O or S; those wherein Y is CH₂; Z is O; and X₁ is N(R_{d}), O or S; and those wherein Y is CH₂; Z is NH; and X₁ is N(R_{d}), O or S.

The present invention also comprises pharmaceutical compositions comprising a therapeutically effective amount of a compound of formula (I) or of formula (II)as defined above or pharmaceutically acceptable salts thereof The present invention also comprises a method of treating a disorder wherein the disorder is ameliorated by inhibiting vanilloid receptor subtype 1 (VR1) receptor in a host mammal in need of such treatment comprising administering a therapeutically effective amount of a compound of formula (I) or a compounds of formula (II) as defined in the foregoing description or pharmaceutically acceptable salts thereof, and wherein the disorder is selected form the group consisting of pain, inflammatory hyperalgesia, bladder overactivity and urinary incontinence.

(2) Definitions

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” means a divalent group derived from a straight or branched chain hydrocarbon of from 2 to 10 carbon atoms containing at least one double bond. Representative examples of alkenylene include, but are not limited to, —CH═CH—, —CH═CH₂CH₂—, and —CH═C(CH₃)CH₂—.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means a phenyl group, or a bicyclic or a tricyclic fuised ring system wherein one or more of the fuised rings is a phenyl group. Bicyclic fused ring systems are exemplified by a phenyl group fuised to a cycloalkyl group, as defined herein, or another phenyl group. Tricyclic fused ring systems are exemplified by a bicyclic fuised ring system fused to a cycloalkyl group, as defined herein, or another phenyl group. Representative examples of aryl include, but are not limited to, anthracenyl, azulenyl, fluorenyl, indanyl, indenyl, naphthyl, phenyl and tetrahydronaphthyl.

The aryl groups of this invention can be substituted with 1, 2, 3, 4 or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, cycloalkyl, cycloalkylalkyl, ethylenedioxy, formyl, formylalkyl, haloalkoxy, haloalkyl, haloalkylthio, halogen, hydroxy, hydroxyalkyl, methylenedioxy, mercapto, mercaptoalkyl, nitro, —NZ_{C}Z_{D}, (NZ_{C}Z_{D})alkyl, (NZ_{C}Z_{D})carbonyl, (NZ_{C}Z_{D})carbonylalkyl, (NZ_{C}Z_{D})sulfonyl, —NR_{A}S(O)₂R_{B}, =13 S(O)₂OR_{A} and —S(O)₂R_{A} wherein R_{A} and R_{B} are as defined herein. The aryl groups ofthis invention can be further substituted with any one of an additional aryl, arylalkyl, aryloxy, arylthio, heterocycle, heterocyclealkyl, heterocycleoxy, or heterocyclethio group, as defined herein, wherein the additional aryl, arylalkyl, aryloxy, arylthio, heterocycle, heterocyclealkyl, heterocycleoxy, and heterocyclethio group can be substituted with 1, 2, 3, 4, or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, —NZ_{C}Z_{D}, (NZ_{C}Z_{D})alkyl, (NZ_{C}Z_{D})carbonyl, (NZ_{C}Z_{D})carbonylalkyl, (NZ_{C}Z_{D})sulfonyl, —NR_{A}S(O)₂R_{B}, —S(O)₂OR_{A} and —S(O)₂R_{A} wherein R_{A} and R_{B} are as defined herein. Representative examples include, but are not limited to, 4-bromophenyl, 3-chlorophenyl, 4-chlorophenyl, 3,4-dichlorophenyl, 2,3-dichlorophenyl, 2,4-dichlorophenyl, 3,5-dichlorophenyl, 3,4-difluorophenyl, 4-bromo-2-fluorophenyl, 4-chloro-2-fluorophenyl, 4-(tert-butyl)phenyl), 4-cyanophenyl, 4-ethylphenyl, 3-fluorophenyl, 2,4-difluorophenyl, 4-bromo-3-fluorophenyl, 2,3-difluoro-4-(trifluoromethyl)phenyl, 3-fluoro4-(trifluoromethyl)phenyl, 3-fluoro-5-(trifluoromethyl)phenyl, 3-(trifluoromethyl)phenyl, 4-(trifluoromethyl)phenyl, 4-(trifluoromethoxy)phenyl, 3-(trifluoromethoxy)phenyl, 4-[(trifluoromethyl)thio]phenyl, 3-methylphenyl, 3,4-dimethylphenyl, 2,4-dimethylphenyl, 4-isopropylphenyl, 4-methylphenyl, 4-bromo-3-methylphenyl, 4-fluoro-3-(trifluoromethyl)phenyl, 3-chloro-4-fluorophenyl, 4-(1-pyrrolidinyl)phenyl, 4-(1-azepanyl)phenyl, 3-fluoro-4-(1-pyrrolidinyl)phenyl, 3-fluoro4-( 1-azepanyl)phenyl, 4-(1-azocanyl)phenyl, 4-(1-piperidinyl)phenyl, 3-fluoro-4-(1-piperidinyl)phenyl, 4-(2-pyridinyl)phenyl, 1,1-biphenyl, 3 -fluoro-4-(4-methyl-1-piperidinyl)phenyl, 4-(4-methyl-1-piperidinyl)phenyl, 4-(4-morpholinyl)phenyl, 4-(2,6-dimethyl-4-morpholinyl)phenyl, 4-(4-thiomorpholinyl)phenyl, 3,5-difluoro4-(4-morpholinyl)phenyl, 3,5-bis(trifluoromethyl)phenyl, and 2,5-bis(trifluoromethyl)phenyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “cycloalkenyl,” as used herein, refers to a non-aromatic, partially unsaturated, monocyclic hydrocarbon ring system, having 4, 5, 6, 7 or 8 carbon atoms and zero heteroatom. The 4-membered ring systems have one double bond, the 5-or 6-membered ring systems have one or two double bonds, and the 7- or 8-membered ring systems have one, two or three double bonds. Representative examples of cycloalkenyl groups include, but not limited to, cyclobutenyl, cyclopentenyl, cyclohexenyl, and octahydronaphthalenyl. The term “cycloalkenyl” of the present invention also include a bicyclic fused ring system wherein the cycloalkenyl ring is fused to a monocyclic cycloalkyl group, as defined herein, or another monocyclic cycloalkenyl group. Representative examples of the bicyclic cycloalkenyl groups include, but not limited to, 4,5,6,7-tetrahydro-3aH-indene and 1,6-dihydro-pentalene. The cycloalkenyl groups of the present invention can be unsubstituted or substituted, and are attached to the parent molecular moiety through any substitutable carbon atom of the group.

The term “cycloalkyl” as used herein, means a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms. Representative examples of bicyclic ring systems include, but are not limited to, bicyclo[3.1.1 ]heptane, bicyclo[2.2.1 ]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms. Representative examples of tricyclic-ring systems include, but are not limited to, tricyclo[3.3.1.0^{3,7}]nonane and tricyclo[3.3.1.1^{3,7}]decane (adamantyl).

The cycloalkyl groups of this invention can be substituted with 1, 2, 3, 4 or 5 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, carboxy, carboxyalkyl, cyano, cyanoalkyl, formyl, haloalkoxy, haloalkyl, haloalkylthio, halogen, hydroxy, hydroxyalkyl, mercapto, mercaptoalkyl, nitro, —NZ_{C}Z_{D}, (NZ_{C}Z_{D})alkyl, (NZ_{C}Z_{D})carbonyl, (NZ_{C}Z_{D})carbonylalkyl, (NZ_{C}Z_{D})sulfonyl, —NR_{A}S(O)₂R_{B}, —S(O)₂OR_{A}, and —S(O)₂R_{A} wherein R_{A} and R_{B} are as defined herein. Representative examples include, but are not limited to, 6,6-dimethylbicyclo[3.1.1 ]heptyl, 6,6-dimethylbicyclo[3.1.1 ]hept-2-yl, 4-tert-butylcyclohexyl, and 4-(trifluoromethyl)cyclohexyl.

The term “formyl” as used herein, means a —C(O)H group.

The term “formylalkyl” as used herein, means a formyl group, as defined herein, appended to the parent molecular moiety through an alkyl group, as defined herein. Representative examples of formylalkyl include, but are not limited to, formylmethyl and 2-formylethyl.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, 2-chloro-3-fluoropentyloxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl” as used herein, refers to an aromatic five- or six-membered ring where at least one atom is selected from the group consisting of N, O, and S, and the remaining atoms are carbon. The five membered rings have two double bonds, and the six membered rings have three double bonds. The term “heteroaryl” also includes bicyclic systems where a heteroaryl ring is fused to a phenyl group, a monocyclic cycloalkyl group, as defined herein, a monocyclic cycloalkenyl group, as defined herein, a heterocycle group, as defined herein, or an additional heteroaryl group. Representative examples of heteroaryl groups include, but not limited to, benzothienyl, benzoxazolyl, benzimidazolyl, benzoxadiazolyl, 6,7-dihydro-benzofuranyl, 6,7-dihydro-1,3-benzothiazolyl, furyl, imidazolyl, imidazo[1,2-α]pyridinyl, indazolyl, indolyl, isoindolyl, isoxazolyl, isoquinolinyl, isothiazolyl, naphthyridinyl, oxadiazolyl, oxazolyl, pyridyl, pyridazinyl, pyrimidinyl, pyrazinyl, pyrazolyl, pyrrolyl, quinolinyl, thiazolyl, thienyl, triazolyl, thiadiazolyl, tetrazolyl, 1,2,3,4-tetrahydro-1,8-naphthyridinyl, 5,6,7,8-tetrahydroquinolinyl and triazinyl. The heteroaryl groups of the present invention can be substituted or unsubstituted, and are connected to the parent molecular moiety through any substitutable carbon or nitrogen atom in the groups. In addition, the nitrogen heteroatom may or may not be quatemized, and may or may not be oxidized to the N-oxide. Also, the nitrogen containing rings may or may not be N-protected.

The term “heterocycle” or “heterocyclic” as used herein, means a monocyclic, bicyclic, or tricyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered ring have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuiranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1 -dioxidothiomorpholinyl (thiomorpholine sulfone), thiopyranyl, triazinyl, triazolyl, and trithianyl. Bicyclic ring systems are exemplified by any ofthe above monocyclic ring systems fused to an aryl group as defined herein, a cycloalkyl group as defined herein, or another monocyclic ring system. Representative examples of bicyclic ring systems include but are not limited to, for example, benzimidazolyl, benzodioxinyl, benzothiazolyl, benzothienyl, benzotriazolyl, benzoxazolyl, benzofuranyl, benzopyranyl, benzothiopyranyl, cinnolinyl, indazolyl, indolyl, 2,3-dihydroindolyl, indolizinyl, naphthyridinyl, isobenzo furanyl, isobenzothienyl, isoindolyl, isoquinolinyl, phthalazinyl, pyranopyridinyl, quinolinyl, quinolizinyl, quinoxalinyl, quinazolinyl, tetrahydroisoquinolinyl, tetrahydroquinolinyl, and thiopyranopyridinyl. Tricyclic rings systems are exemplified by any of the above bicyclic ring systems fused to an aryl group as defined herein, a cycloalkyl group as defined herein, or a monocyclic ring system. Representative examples oftricyclic ring systems include, but are not limited to, acridinyl, carbazolyl, carbolinyl, dibenzo[b,d]furanyl, dibenzo[b,d]thienyl, naphtho[2,3-b]furan, naphtho [2,3 -b]thienyl, phenazinyl, phenothiazinyl, phenoxazinyl, thianthrenyl, thioxanthenyl and xanthenyl. The monocyclic, bicyclic and tricyclic heterocycles of the present invention may have two of the non-adjacent carbon atoms connected by a heteroatom selected from N, N(H), O or S, or an alkylene bridge of between one and three additional carbon atoms. The heterocycles of this invention can be substituted with 1, 2,or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcarbonyloxy, alkylsulfonyl, alkylthio, alkynyl, arylalkyl, aryloxy, arylthio, carboxy, carboxyalkyl, cyano, cyanoalkyl, cycloalkyl, cycloalkylalkyl, formyl, formylalkyl, haloalkoxy, haloalkyl, haloalkylthio, halogen, hydroxy, hydroxyalkyl, mercapto, mercaptoalkyl, nitro, oxo, —NZ_{C}Z_{D}, (NZ_{C}Z_{D})alkyl, (NZ_{C}Z_{D})carbonyl, (NZ_{C}Z_{D})carbonylalkyl, (NZ_{C}Z_{D})sulfonyl, —NR_{A}S(O)₂R_{B}, —S(O)₂OR_{A} and —S(O)₂R_{A} wherein R_{A} and R_{B} are as defined herein. The heterocycles of this invention can be fuirther substituted with any one of an additional aryl, arylalkyl, aryloxy, arylthio, heterocycle, heterocyclealkyl, heterocycleoxy, or heterocyclethio group, as defined herein, wherein the additional aryl, arylalkyl, aryloxy, arylthio, heterocycle, heterocyclealkyl, heterocycleoxy, and heterocyclethio group can be substituted with 1, 2, or 3 substituents independently selected from alkenyl, alkoxy, alkoxyalkoxy, alkoxyalkyl, alkoxycarbonyl, alkoxycarbonylalkyl, alkyl, alkylcarbonyl, alkylcarbonylalkyl, alkylcaibonyloxy, alkylsulfonyl, alkylthio, alkynyl, carboxy, carboxyalkyl, cyano, cyanoalkyl, cycloalkyl, cycloalkylalkyl, ethylenedioxy, formyl, formylalkyl, haloalkoxy, haloalkyl, haloalkylthio, halogen, hydroxy, hydroxyalkyl, mercapto, mercaptoalkyl, nitro, —NZ_{C}Z_{D}, (NZ_{C}Z_{D})alkyl, (NZZ_{D})carbonyl, (NZ_{C}Z_{D})carbonylalkyl, (NZ_{C}Z_{D})sulfonyl, —NR_{A}S(O)₂R_{B}, —S(O)₂OR_{A} and —S(O)₂R_{A} wherein R_{A} and R_{B} are as defined herein. Representative examples include, but are not limited to, 8-azabicyclo[3.2.1]oct-8-yl, azepan-1-yl, 2,6-dimethylmorpholinyl, 4-(3-chlorophenyl)-1-piperazinyl, 4-(3,4-dimethylphenyl)-1-piperazinyl, 4-(4-chlorophenyl)-1-piperazinyl, 4-(4-methylphenyl)-3-methyl-1-piperazinyl, 4-(2,3-dimethylphenyl)-1-piperazinyl, 4-(2,3-dichlorophenyl)-1-piperazinyl, 4-(3,4-dichlorophenyl)-1-piperazinyl, 4-[3-(trifluoromethyl)phenyl]-1-piperazinyl, 4-(4-bromophenyl)-1-piperazinyl, 2-oxo-1-pyrrolidinyl, and 5-(trifluoromethyl)-2-pyridinyl.

The term “hydroxy” as used herein, means an —OH group.

The term “hydroxyalkyl” as used herein, means at least one hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “nitro” as used herein, means a —NO₂ group.

The term “—NR_{d}Rₑ” as used herein, means two groups, R_{d} and Rₑ, which are appended to the parent molecular moiety through a nitrogen atom. R_{d} and Rₑ are each independently selected from hydrogen, alkyl, alkylcarbonyl, formyl, aryl and arylalkyl. Representative examples of —NR_{d}Rₑ include, but are not limited to, amino, methylamino, acetylamino, benzylamino, phenylamino, and acetylmethylamino. The term “(“—NR_{d}Rₑ)alkyl” as used herein, means a —”—NR_{d}Rₑ group, as defined herein, appended to the parent molecular moiety through an alkyl group, as defined herein. Representative examples of (“—NR_{d}Rₑ)alkyl include, but are not limited to, aminomethyl, 2-(methylamino)ethyl, 2-(dimethylamino)ethyl and (ethylmethylamino)methyl.

(3) Schemes

The compounds and processes of the present invention will be better understood in connection with the following synthetic schemes, which illustrate the methods by which the compounds ofthe invention may be prepared. Starting materials can be obtained from commercial sources or prepared by well-established literature methods known to those of ordinary skill in the art. The groups R₁, R_{4,} and R_{5,} are as defined above unless otherwise noted below.

This invention is intended to encompass compounds having formula (I) or (II) when prepared by synthetic processes or by metabolic processes. Preparation ofthe compounds of the invention by metabolic processes includes those occurting in the human or animal body (in vivo) or processes occurring in vitro.

If a substituent described herein is not compatible with the synthetic methods ofthis invention, the substituent may be protected with a suitable protecting group that is stable to the reaction conditions used in these methods. The protecting group may be removed at suitable point in the reaction sequence of the method to provide a desired intermediate or target compound. Suitable protecting groups and the methods for protecting and deprotecting different substituents using such suitable protecting groups are well know to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety.

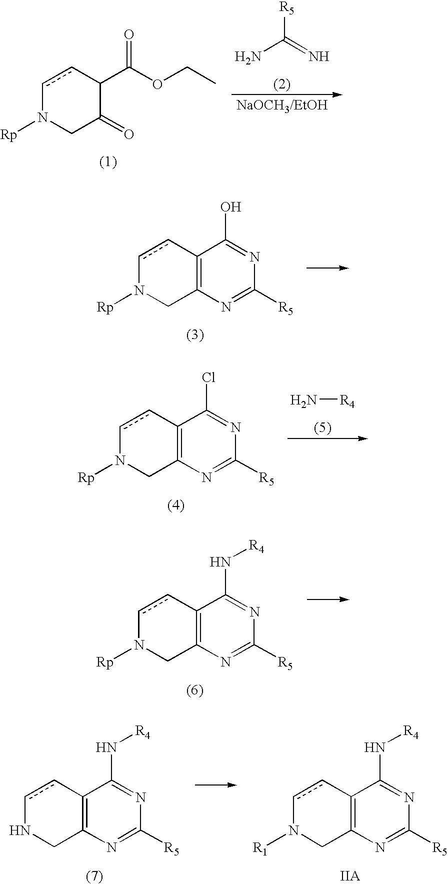

Compounds of formula (IIA), wherein z,900 is a single bond or a double bond, Rp is a nitrogen protecting group such as diphenylmethyl, substituted diphenylmethyl (for example, bis(4-methoxyphenyl)methyl and the like), benzyl or substituted benzyl (for example, 4-methoxybenzyl, 2,4-dimethoxybenzyl, and the like) and R₁, R₄ and R₅ are as defined herein can be prepared as shown in Scheme 1. Acid salts of compounds of formula (1), either purchased or prepared by methodologies well known by those skilled in the art, can be converted to compounds of formula (3) by reaction with acid salts of amidines having formula (2), in the presence of about two equivalents of a base. The reaction is generally performed in a solvent such as, but is not limited to, alcoholic solvents such as ethanol or methanol, dichloromethane, tetrahydrofuiran, ethyl acetate, or acetone at a temperature between about room temperature to about 100° C. for a period of about an hour to about 2 days. Examples ofthe base include, but are not limited to, metal alkoxides such as sodium ethoxide, organic bases such as, but are not limited to, triethylamine, pyridine and 1-methylimidazole, and hydroxides or carbonates of alkali metals such as lithium, sodium, potassium.

Conversion of compounds of formula (3) to compounds of formula (4) can be achieved heating in phosphorous oxychloride at a temperature from about 50° C. to about 100° C., for a period of about 1 hour to about 1 day.

Compounds of formula (6) can be obtained by microwave irradiation or heating of compounds of formula (4) with an amine of formula (5) at a temperature between about 180 to about 200° C., in the presence of pyridine, for a period of about 15 minutes to about 1 hour.

Compounds of formula (6) can be deprotected using procedures that are well known in the art. For example, compounds of formula (6) wherein Rp is benzyl can be deprotected by catalytic hydrogenation, to afford compounds of formula (7). The reaction can be effected with hydrogen gas (H₂), using catalysts such as palladium on carbon (Pd/C), platinum on carbon (Pt/C) or palladium hydroxide on carbon (Pd(OH)_{2/}C), with or without acetic acid, in an appropriate solvent such as, but not limited to, methanol, ethanol, tetrahydroftiran, dioxane or ethyl acetate, or mixture thereof, at a pressure from about 1 to about 5 atmospheres and a temperature between about 10° C. to about 60° C. An alternative procedure employing the use of reagents such as ammonium formate and Pd/C in methanol at reflux temperature under an inert atmosphere (e.g. nitrogen gas) is also effective.

Compounds of formula IIA can be obtained from compounds of formula (7) by microwave irradiation or heating with potassium carbonate and compounds of formula R₁X wherein X is Cl, Br or I, at a temperature between about 150° C to about 200° C., in an appropriate solvent such as dimethyl sulfoxide, N,N-dimethylacetamide, N-methylpyrrolidinone N,N-dimethylformamide, for a period of about 5 minutes to about 1 hour. Alternatively, compounds of formula IIA can be prepared by reaction of compounds of formula (7) with compounds of formula RIX, wherein X is halide in the presence of a suitable base and a suitable catalyst. Suitable bases include alkali metal carbonates or hydroxide bases, preferably potassium carbonate. Suitable catalysts include copper (0), copper (I) or palladium ligands catalyst, preferably finely powdered copper bronze. Suitable solvents for the aforesaid reaction include neat or polar aprotic solvents, such as but not limited to, dimethyl sulfoxide, N,N-dimethylacetamide, and N-methylpyrrolidinone N,N-dimethylformamide. The reaction may be run at a temperature between about 80° C. to about 190° C. for about 6 to 24 hours.

### 4) EXAMPLES

It is understood that the following Examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof

### Example 1

7-benzyl-N-(4-tert-butylphenyl)-5,6,7,8-tetrahydropyrido[3,4-d]pyrimidin-4-amine

### Example 1A

7-Benzyl-4-Chloro-5,6,7,8-Tetrahydropyrido[3,4-d]Pyrimidine

A mixture of ethyl 1-benzyl-3-oxopiperidine-4-carboxylate hydrochloride (6.10 g, 20.5 mmol), formamidine hydrochloride (Aldrich, 1.65 g, 20.5 mmol) and sodium ethoxide (2.7 M in ethanol, 18 mL, 48 mmol) in ethanol (54 mL) was heated to 60° C. and stirred overnight. The mixture was cooled to ambient temperature, concentrated, diluted with water and extracted with dichloromethane. The organic layer was dried (Na₂SO₄), filtered and concentrated. The concentrate was heated in phosphorus oxychloride (Aldrich, 50 mL) at 90° C. for 3 hr. The mixture was cooled to about 25° C., concentrated, diluted with saturated, aqueous NaHCO₃, and extracted with dichloromethane. The organic layer was dried (Na₂SO₄), filtered, concentrated, and purified by flash chromatography, eluted with 25% diethyl ether in hexanes to give the title compound.

### Example 1B

7-Benzyl-N-(4-Tert-Butylphenyl)-5,6,7,8-Tetrahydropyrido[3,4-d]Pyrimidin-4-Amine

A solution of Example 1A (0.744 g, 2.86 mmol), 4-tert-butylaniline (0.55 mL, 3.5 mmol), and pyridine (0.35 mL, 4.3 mmol) in tetrahydrofuran (2.9 mL) was microwave-irradiated at 180° C. for 15 min. The mixture was cooled to about 25° C., diluted with saturated, aqueous NaHCO₃, extracted with dichloromethane, dried (Na₂SO₄), filtered and concentrated. The concentrate was chromatographed on silica gel, eluting with diethyl etherto give the title compound. ¹H NMR (300 MHz, CD₃OD) δ 8.24 (s, 1H), 7.26-7.49 (m, 9H), 3.75 (s, 2H), 3.52 (s, 2H), 2.87 (t, J=5.8 Hz, 2H), 2.67 (t, J=5.8 Hz, 2H), 1.32 (s, 9H). MS (m/z) 373.

### Example 2

N-(4-Tert-Butylphenyl)-5,6,7,8 -Tetrahydropyrido [3,4-d]Pyrimidin-4-Amine

A mixture of Example 1B (0.625 g, 1.68 mmol) and 20% Pd(OH)₂/C (0.2 g) in methanol (25 mL) was shaken under H₂ (65 psi) for 3 hr, filtered, concentrated, and chromatographed on silica gel, eluted with 2% triethylamine in 8% methanol/dichloromethane to give the title compound. ¹H NMR (300 MHz, CD₃0D): δ 8.26 (s, 1H), 7.46 (d, J=6.8 Hz, 2H), 7.39 (d, J=6.8 Hz, 2H), 3.83 (s, 2H), 3.18 (t, J=5.8 Hz, 2H), 2.63 (t, J=5.8 Hz, 2H), 1.33 (s, 9H). MS (m/z) 283.

### Example 3

N-(4-Tert-Butylphenyl)-7-(3-Chloropyridin-2-yl)-5,6,7,8-Tetrahydropyrido[3,4-d]Pyrimidin-4-Amine

A mixture of Example 2 (65.2 mg, 0.231 mmol), 2,3-dichloropyridine (41.1 mg, 0.359 mmol), and K₂CO₃ (64.6 mg, 0.467 mmol) in dimethylsulfoxide (0.50 mL) was microwave-irradiated at 200° C. for 15 min, cooled to about 25° C., diluted with water, and extracted with dichloromethane. The organic extract was dried (Na₂SO₄), filtered and concentrated. The concentrate was chromatographed on silica gel, eluted with diethyl ether to give the title compound. ¹H NMR (300 MHz, CDCl₃): δ 8.58 (s, 1H),8.20 (dd, 1H), 7.63 (dd, 1IH), 7.88 (d, 2H), 7.41 (d, 2H), 6.88 (dd, 1IH), 6.45 (brs, 1lH), 4.58 (s, 2H), 3.80 (t, 2H), 2.80 (t, 2H), 1.33 (s, 9H). MS (m/z) 394.

### Example 4

N-(4-Tert-Butylphenyl)-7-Pyrinidin-2-yl-5,6,7,8-Tetrahydropyrido[3,4-d]Pyvrimidin4-Amine

A mixture of Example 2 (65.2 mg, 0.231 mmol), 2-chloropyrimidine (41.1 mg, 0.359 mmol), and K₂CO₃ (64.6 mg, 0.467 mmol) in dimethylsulfoxide (0.50 mL) was microwave-irradiated at 200° C. for 15 min, cooled to about 25° C., diluted with water, and extracted with dichloromethane. The organic extract was dried (Na₂SO₄), filtered and concentrated. The concentrate was chromatographed on silica gel, eluted with diethyl ether to give the title compound. ¹H NMR (300 MHz, CDCl₃): δ 8.59 (s, 1H), 8.36 (d, 2H), 7.46 (d, 2H), 7.41 (d, 2H), 6.57 (t, 1H), 5.01 (s, 2H), 4.24 (t, 2H), 2.69 (t, 2H), 1.33 (s, 9H). MS (m/z) 361.

### Example 5

N-(4-Tert-Butylphenyl)-7-[3-(Trifluoromethyl)Pyridin-2-yl]-5,6,7,8-Tetrahydropyrido [3,4-5 d]Pyrimidin4-Amine

The title compound was prepared using the procedure as described in Example 4, substituting 2-chloro-3-(trifluoromethyl)pyridine for 2-chloropyrimidine. ¹H NMR (300 MHz, CDCl₃): δ 8.58 (s, 1H), 8.47 (dd, 1H), 7.91 (dd, 1H), 7.49 (d, 2H), 7.41 (d, 2H), 7.05 dd, 1H), 6.40 (brs, 1H), 4.50 (s, 2H), 3.71 (t, 2H), 2.77 (t, 2H), 1.33 (s, 9H). MS (m/z) 428.

### Example 6

2-[4-[(4-Tert-Butylphenyl)Amino]-5,8-Dihydropyridor[3,4-d]Pyrimidin-7(6H)-yll-N,N-Dimethylpyridine-3 -Sulfonamide

The title compound was prepared using the procedure as described in Example 4, substituting 2-chloro-N,N-dimethylpyridine-3 -sulfonamide for 2-chloropyrimidine. ¹H NMR (300 MHz, CDCl₃) δ 8.58 (s, 1 H), 8.49 (dd, 1 H), 8.22 (dd, 1 H), 7.51 (d, 2H), 7.41 (d,. 2H), 7.16 (dd, 1H), 6.43 (brs, 1H), 4.48 (s, 2H), 3.69 (t, 2H), 2.84 (t, 2H), 2.71 (s, 6H), 1.33 (s, 9H). MS (m/z) 467.

### Example 7

N-(4-Tert-Butylphenyl)-2-Methyl-7-[3-(Trifluoromethyl)Pyridin-2-yl]-5,6,7,8-Tetrahydropyrido[3,4-d]Pyrimidin-4-Amine

### Example 7A

7 -Benzyl4 -Chloro-2 -Methyl-5,6,7,8 -Tetrahydropyrido[3,4-d]Pyrimidine

A mixture of ethyl 1-benzyl-3-oxopiperidine-4-carboxylate hydrochloride (0.512 g, 1.72 mmol), acetamidine hydrochloride (Aldrich, 0.166 g, 1.75 mmol) and sodium ethoxide (2.7 M in ethanol, 1.5 mL, 4.0 mmol) in ethanol (4.5 mL) was heated to 60° C. and stirred overnight. The mixture was cooled to about 25° C., concentrated, diluted with water and extracted with dichloromethane. The organic layer was dried (Na₂SO₄), filtered, and concentrated. The concentrate was heated in phosphorus oxychloride (Aldrich, 50 mL) at 90° C. for 5 hr. The mixture was cooled to about 25° C., concentrated, diluted with saturated, aqueous NaHCO₃, and extracted with dichloromethane. The organic layer was dried (Na₂SO₄), filtered and concentrated to give the title compound.

### Example 7B

7-Benzyl-N-(4-Tert-Butylphenyl)-2-Methyl-5,6,7,8-Tetrahydropyrido[3,4-d]Pyrimidin-4-Amine

A solution of Example 7A (0.207 g, 0.755 mmol), 4-tert-butylaniline (0.15 mL, 0.94 mmol), and pyridine (0.12 mL, 1.5 mmol) in tetrahydrofuran (2.5 mL) was microwave-irradiated at 180° C. for 15 min. The mixture was cooled to 25° C., diluted with saturated, aqueous NaHCO₃, extracted with dichloromethane, dried (Na₂SO₄), filtered and concentrated. The concentrate was chromatographed on silica gel, eluted with 3% methanol in dichloromethane to give the title compound.

### Example 7C

N-(4-Tert-Butylphenyl)-2-Methyl-5,6,7,8-Tetrahydropyrido[3,4-d]Pyrimidin-4-Amine

A mixture of Example 7B (0.207 g, 0.535 mmol) and 20% Pd(OH)₂/C (0.2 g) in methanol (7 mL) was shaken under H₂ (65 psi) for 5 hr, filtered and concentrated to give the title compound.

### Example 7D

N-(4-tert-butylphenyl)-2-methyl-7-[3-Ctrifluoromethyl)pyridin-2-yl]-5,6,7 8,-tetrahydropyrido[3,4-d]pyrimidin-4-amine

The title compound was prepared using the procedure as described in Example 4, substituting Example 7C for Example 2 and substituting 2-chloro-3-trifluoromethylpyridine for 2-chloropyrimidine. ¹H NMR (300 MHz, CDCl₃): δ 8.44 (m, 1H),7.90 (d, 1), 7.57 (d, 2H), 7.39 (d, 2H), 7.02 (m, 1H), 6.32 (brs, 1H), 4.46 (s, 2H), 3.70 (t, 2H), 2.72 (t, 2H), 2.58 (s, 3H), 1.33 (m, 9H). MS (m/z) 442.

### Example 8

N-(4-Tert-Butylphenyl)-2-Phenyl-7-[3-(Trifluoromethyl)1Pridin-2-yl]-5,6,7,8-Tetrahydropyrido[3 ,4-d]Pydrinid-4-Amine

### Example 8A

7-Benzyl4-Chloro-2 -Phenyl-5,6,7,8 -Etrahydropyrido[3 4-d]Pyrimidine

A mixture of ethyl 1 -benzyl-3-oxopiperidine-4-carboxylate hydrochloride (0.483 g, 1.62 mmol), benzamidine hydrochloride (Aldrich, 0.254 g, 1.62 mmol) and sodium ethoxide (2.7 M in ethanol, 1.5 mL, 4.0 mmol) in ethanol (4.5 mL) was heated to 60° C. and stirred overnight. The mixture was cooled to about 25° C., concentrated, diluted with water and extracted with dichloromethane. The organic layer was dried (Na₂SO₄), filtered and concentrated. The concentrate was heated in phosphorus oxychloride (Aldrich, 50 mL) at 90° C. for 5 hr. The mixture was cooled to about 25° C., concentrated, diluted with saturated, aqueous NaHCO₃, and extracted with dichloromethane. The organic layer was dried (Na₂SO₄), filtered and concentrated to give the title compound.

### Example 8B

7-Benzyl-N-(4-Tert-Butylphenyl)-2-Phenyl-5,6,7,8-Tetrahydropyrido[3,4-d]Pyrimidin4-Amine

A solution of Example 8A (0.467 g, 0.1.39 mmol), 4-tert-butylaniline (0.27 mL, 1.7 mmol), and pyridine (0.17 mL, 2.1 mmol) in tetrahydrofuran (4.0 mL) was microwave-irradiated at 180° C. for 15 min. The mixture was cooled to about 25° C., diluted with saturated, aqueous NaHCO₃, extracted with dichloromethane, dried (Na2SO4), filtered, and concentrated. The concentrate was chromatographed on silica gel, eluted with 75% diethyl ether in hexanes to give the title compound.

### Example 8C

N-(4-Tert-Butylphenyl)-2-Phenyl-5,6,7,8-Tetrahydropyrido[3.4-d]Pyrimidin-4-Amine

A mixture of Example 8B (0.519 g, 1.16 mmol) and 20% Pd(OH)₂JC (0.3 g) in methanol (10 mL) and ethyl acetate (10 mL) was shaken under H₂ (65 psi) overnight. More catalyst (0. 15 g) and acetic acid (0.3 mL) were added, and the mixture was stirred under H₂ (65 psi) for 3 hr. The mixture was filtered, concentrated, and chromatographed on silica gel, eluted with 2% triethylamine in 7% methanol/dichloromethane to give the title compound.

### Example 8D

N-(4-Tert-Butylphenyl)-2-Phenyl-7-[3-(Trifluoromethyl)Pyridin-2-yl]-5,6,7,8-Tetrahydropyrido[3,4-d]Pyrimidin-4-Amine

A mixture ofthe Example 8C (0.145 g, 0.401 mmol), 2-chloro-3-trifluoromethylpyridine (0.112 g, 0.619 mmol), and K₂CO₃ (0.111 g, 0.804 mmol) in DMSO (0.8 mL) was microwave-irradiated at 190° C. for 20 min, cooled to about 25° C., diluted with water, and extracted with dichloromethane. The organic extract was dried (Na₂SO₄), filtered and concentrated. The concentrate was chromatographed on silica gel, eluting with 33% diethyl ether in hexanes to give the title compound. ¹H NMR (300 MHz, CDCl₃) δ 8.46 (d, 1H), 8.42 (brs, 2H), 7.91 (dd, 1H), 7.68 (d, 2H), 7.41-7.49 (m, 5H), 7.03 (dd, 1H), 6.41 (brs, 1H), 4.58 (s, 2H), 3.74 (t, 2H), 2.81 (t, 2H), 1.36 (s, 9H).

### Example 9

N-(4-tert-butylphenyl)-7-(3-chloropvridin-2-yl)-2-phenyl-5 6,7,8-tetrahydropyrido[3,4-d]pyrimidin4-amine

The title compound was prepared using the procedure as described in Example 8D, substituting 2,3-dichloropyridine for 2-chloro-3-trifluoromethylpyridine. ¹H NMR (300 MHz, CDCl₃) δ 8.42 (m, 2H), 8.21 (dd, 1H), 7.69 (d, 2H), 7.63 (dd, 1H), 7.41-7.50 (m, 5H), 6.88 (dd, 1H), 6.40 (brs, 1H), 4.63 (s, 2H), 3.83 (t, 2H), 2.84 (t, 2H), 1.36 (s, 9H). MS (m/z) 470.

### Example 10

2-Tert-Butyl-N-(4-Tert-Butylphenyl)-7-[3-(Trifluoromethyl)Pyridin-2-yl]-5,6,7,8-Tetrahydropyrido[3,4-d]Pyrimidin-4-Amine

### Example 10A

7-Benzyl-2-Tert-Butyl4-Chloro-5,6,7,8-Tetrahydropyrido[3,4-d]Pyrimidine

A mixture of ethyl 1-benzyl-3-oxopiperidine-4-carboxylate hydrochloride (4.36 g, 14.6 mmol), t-butylcarbamidine hydrochloride (2.00 g, 14.6 mmol) and sodium ethoxide (2.7 M in ethanol, 12.5 mL, 33.8 mmol) in ethanol (38 mL) was heated to 60° C. and stirred overnight. The mixture was cooled to about 25° C., concentrated, diluted with water and extracted with dichloromethane. The organic layer was dried (Na₂SO₄), filtered and concentrated. The concentrate was heated in phosphorus oxychloride (Aldrich, 50 mL) at 90° C. for 3 hr. The mixture was cooled to 25° C., concentrated, diluted with saturated, aqueous NaHCO₃, and extracted with dichloromethane. The organic layer was dried (Na₂SO₄), filtered, concentrated, and filtered through SiO₂ with 25% diethyl ether in hexanes to give the title compound.

### Example 10B

7-Benzyl-2-Tert-Butyl-N-(4-Tert-Butylphenyl)-5,6,7,8-Tetrahedropyrido[3,4-d]Pyrimidin-4-Amine

A solution of Example 10A (0.800 g, 2.54 mmol), 4-tert-butylaniline (0.48 mL, 3.0 mmol), and pyridine (0.30 mL, 3.8 mmol) in tetrahydrofuran (8.0 mL) was microwave-irradiated at 180° C. for 25 min. The mixture was cooled to 25° C., concentrated, diluted with saturated, aqeous NaHCO₃, and extracted with dichloromethane. The organic phase was dried (Na₂SO₄), filtered, concentrated, and chromatographed on silica gel, eluted with 50% diethyl ether in hexanes to give the title compound.

### Example 10C

2-Tert-Butyl-N-(4-Tert-Butylphenyl)-5,6,7,8-Tetrahydropyrido[3,4-d]Pyrimidin4-Amine

A mixture of Example 10B (0.519 g, 1.16 mmol) and 20% Pd(OH)₂/C (0.3 g) in methanol (12 mL) and ethyl acetate (3 mL)was shaken under H₂ (65 psi) overnight, filtered, and concentrated to give the title compound.

### Example 10D

2-Tert-Butyl-N-(4-Tert-Butylphenyl)-7-[3-(Trifluoromethyl)Pyridin-2-yl]-5 6,7,8-Tetrahydropyrido[3,4-d]Pyrimidin4-Amine

A mixture of the Example 10C (0.242 g, 0.714 mmol), 2-chloro-3-trifluoromethylpyridine (0.195 g, 1.08 mmol), and K₂CO₃ (0.199 g, 1.44 mmol) in dimethylsulfoxide (1.4 mL) was microwave-irradiated at 190° C. for 20 min, cooled to about 25° C., diluted with water, and extracted with dichloromethane. The organic extract was dried (Na₂SO₄), filtered and concentrated. The concentrate was chromatographed on silica gel and eluted with 25% diethyl ether in hexanes. The product obtained was triturated with hexanes to give the title compound. ¹H NMR (300 MHz, DMSO-d₆) δ 8.53 (d, 1H), 8.29 (s, 1H), 8.11 (dd, 1H), 7.78 (d, 2H), 7.33 (d, 2H), 7.19 (dd, 1H), 4.30 (s, 2H), 3.61 (t, 2H), 2.77 (t, 2H), 1.30 (s, 9H), 1.28 (s, 9H). MS (m/z) 484.

### Example 11

2-Tert-Butyl-N-(4-Tert-Butylphenyl)-7-(3-Chloropyridin-2-yl)-5,6,7,8-Tetrahydropyrido[3,4-d]Pyrimidin4-Amine

The title compound was prepared using the procedure as described in Example 10D, substituting 2,3-dichloropyridine for 2-chloro-3-trifluoromethylpyridine. ¹H NMR (300 MHz, CDCl₃) δ 8.20 (dd, 1H), 7.69 (d, 2H), 7.60 (dd, 1H), 7.38 (d, 2H), 6.86 (dd, 1H), 6.29 (s, 1H), 4.51 (s, 2H), 3.77 (t, 2H), 2.78 (t, 2H), 1.39 (s, 9H), 1.34 (s, 9H). MS (m/z) 450.

### Example 12

2-Tert-Butyl-N-(4-Tert-Butylphenyl)-7-(1,3-Thiazol-2-yl)-5,6,7,8-Tetrahydropyrido[3,4-d]Pyrimidin4-Amine

The title compound was prepared using the procedure as described in Example 10D, substituting 2-bromothiazole for 2-chloro-3-trifluoromethylpyridine. ¹H NMR (300 MHz, CDCl₃) δ 7.65 (d, 2H), 7.38 (d, 2H), 7.24 (d, 1H), 6.64 (d, 1H), 6.32 (brs, 1H), 4.52 (s, 2H), 4.03 (t, 2H), 2.72 (t, 2H), 1.41 (s, 9H), 1.34 (s, 9H). MS (m/z) 422.

### Example 13

N-(4-Tert-Butylphenyl)-7-(1,3-Thiazol-2-yl)-5,6,7,8-Tetrahydropvrido[3,4-d]Pyrimidin4-Amine

The title compound was prepared using the procedure as described in Example 4, substituting 2-bromothiazole for 2-chloropyiimidine. ¹H NMR (300 MHz, CDCl₃) δ 1.33 (s, 9 H), 2.77 (t, J=5. 1 Hz, 2 H), 4.08 (t, J=4.7 Hz, 2 H), 4.57 (s, 2 H), 6.41 (s, 1 H), 6.65 (d, J=3.7 Hz, 1 H), 7.25 (s, 1 H), 7.40 (m, 2 H), 7.47 (m, 2 H), 8.58 (s, 1 H). MS (m/z) 366 (M+H)⁺

### Example 14

N-(4-Azepan-1-Ylphenyl)-7-Pyrimidin-2-yl-5,6,7,8-Tetrahydropyrido [3,4-d]Pyrimidin4-Amine

### Example 14A

7-Benzyl-N-(4-Azepan-1-yl-Phenyl)-5,6,7,8-Tetrahydropyrido[3,4-d]Pyrimidin4-Amine

A solution of Example 1A (0.520 g, 2.0 mmol), 4-azepanylaniline (0.494 g, 2.6 mmol), and pyridine (0.243 g, 3.0 mmol) in tetrahydrofuran (4.0 mL) was microwave-irradiated at 180° C. for 15 min. The mixture was cooled to 25° C., concentrated, and chromatographed on silica gel, eluted with 5% methanol in dichloromethane to give the title compound.

### Example 14B

N-(4-Azepan-1-yl-Phenyl)-5,6,7,8-Tetrahydropyrido[3 4-d]Pyrimidin4-Amine

A mixture of Example 14A (0.400 g, 0.97 mmol) and 20% Pd(OH)_{2/}C (0.12 g) in methanol (25 mL) and acetic acid (0.3 mL) was shaken under H₂ (60 psi) overnight, treated with more 20% Pd(OH)₂/C (0.12 g), heated to 50° C., shaken under H₂ (60 psi) for 16 hours, cooled, filtered, and concentrated to give the title compound.

### Example 14C

N-(4-Azepan-1-Ylphenyl)-7-Pyrimidin-2 -yl-5,6,7,8 -Tetrahydropyrido [3,4-d]Pyrimidin4-Amine

A mixture of Example 14B (97 mg, 0.30 mmol), 2-chloropyrimidine (49 mg, 0.33 mmol), and K₂CO₃ (83 mg, 0.60 mmol) in dimethylsulfoxide (1.0 mL) was microwave-irradiated at 200° C. for 10 min, cooled to about 25° C., diluted with water, and extracted with dichloromethane. The organic extract was dried (Na₂SO₄), filtered and concentrated. The concentrate was chromatographed on silica gel, eluted with 5% methanol in dichloromethane to give the title compound. ¹H NMR (300 MHz, DMSO-d₆): δ 1.47 (m,4 H), 1.73 (s, 4 H), 2.65 (t, J=5.8 Hz, 2 H), 3.43 (t, J=5.9 Hz, 4 H), 4.10 (t, J=5.8 Hz, 2 H), 4.70 (s, 2 H), 6.63 (d, J=9.2 Hz, 2 H), 6.69 (t, J=4.7 Hz, 1 H), 7.30 (d, J=9.2 Hz, 2 H), 8.26 (m, 2 H), 8.43 (d, J=4.7 Hz, 2 H). MS (m/z) 402.

### Example 15

N-[4-(8-Azabicyclo [3.2.1]Oct-8-yl)-3 -Fluorophenyl]-7 -[3 -(Trifluoromethylpyridin-2 -yl]-5,6,7,8-Tetrahydropyrido[3 4-d]Pyrimidin4-Amine

### Example 15A

7-Benzyl-N-(4-(8-Aza-Bicyclo[3.2.1Oct-8-yl)-3-Fluoro-Phenyl)-5,6,7,8-Tetrahydropvrido[3,4-d]Pyrimidin-4-Amine

A solution of Example 1A (0.520 g, 2.0 mmol), 4-(8-aza-bicyclo[3.2.1]oct-8-yl)-3-fluoroaniline (0.525 g, 2.4 mmol), and pyridine (0.24 g, 3.0 mmol) in tetrahydrofuran (4.0 mL) was microwave-irradiated at 180° C. for 15 min. The mixture was cooled to 25° C., concentrated, and chromatographed on silica gel, eluted with 5% methanol in dichloromethane to give the title compound.

### Example 15B

N-(4-(8-Aza-Bicyclo[3.2.1]Oct-8-yl)-3-Fluorophenyl)-5,6,7,8-Tetrahydropyrido[3,4-d]Pyrimidin4-Amine

A mixture of Example 15A (0.446 g, 1.0 mmol) and 20% Pd(OH)₂/C (0.12 g) in methanol (25 mL) and acetic acid (0.3 mL) was shaken under H₂ (60 psi) overnight, filtered, and concentrated to give the title compound.

### Example 15C

N-[4-(8-Azabicyclo [3.2.1 Oct-8-yl)-3 -Fluorophenyl]-7-[3-(Rifluoromethyl)Pyridin-2-yl]-5,6,7,8-Tetrahydropyrido[3,4-d]Pyrimidin4-Amine

A mixture of Example 15B (106 mg, 0.30 mmol), 2-chloro-3-trifluoromethylpyridine (60 mg, 0.33 mmol), and K₂CO₃ (83 mg, 0.60 mmol) in dimethylsulfoxide (1.0 mL) was microwave-irradiated at 200° C. for 10 min, cooled to about 25° C., diluted with water, and extracted with dichloromethane. The organic extract was dried (Na₂SO₄), filtered and concentrated. The concentrate was chromatographed on silica gel, eluted with 5% methanol in dichloromethane to give the title compound. ¹H NMR (300 MHz, DMSO-d₆): δ 1.39 (m, 3 H), 1.75 (m, 5 H), 1.94 (m, 2 H), 2.76 (t, J=5.4 Hz, 2 H), 3.60 (t, J=5.6 Hz, 2 H), 4.11 (s, 2 H), 4.30 (s, 2 H), 6.92 (t, J=10.2 Hz, 1 H), 7.21 (dd, J=7.8, 4.7 Hz, 1 H), 7.31 (m, 1 H), 7.54 (dd,J=16.1, 2.5 Hz, 1 H), 8.12 (dd,J=8.0, 1.9 Hz, 1 H), 8.39 (m, 2 H), 8.55 (dd, J=4.2, 1.5 Hz, 1H). MS (m/z) 499.

### Example 16

N-[4-(8-Azabicyclo[3.2.1]Oct-8 )-3-Fluorophenyl]-7-Pyrimidin-2-yl-5,6,7,8-Tetrahydropyrido[3,4-d]Pyrimidin-4-Amine

A mixture of Example 15B (106 mg, 0.30 mmol), 2-chloropyrimidine (49 mg, 0.33 mmol), and K₂CO₃ (83 mg, 0.60 mmol) in dimethylsulfoxide (1.0 mL) was microwave-irradiated at 200° C. for 10 min, cooled to about 25° C., diluted with water, and extracted with dichloromethane. The organic extract was dried (Na₂SO₄), filtered and concentrated. The concentrate was chromatographed on silica gel, eluted with diethyl ether to give the title compound. ¹H NMR (300 MHz, DMSO-d₆): δ 1.40 (m,3 H), 1.75 (m,5 H), 1.93 (m,2 H), 2.69 (t,J=5.8 Hz, 2 H), 4.10 (m, 4 H), 4.73 (s, 2 H), 6.69 (t,J=4.7 Hz, 1 H), 6.91 (m, 1 H), 7.29 (dd, J=8.6, 2.2 Hz, 1 H), 7.52 (dd, J=16.1, 2.5 Hz, 1 H), 8.40 (m, 2 H), 8.43 (d, J=4.7 Hz, 2 H). MS (m/z) 432.

### Example 17

N-[4-(Trifluoromethyl)Phenyl]-7-[3-(Trifluoromethyl)Pyridin-2-yl]-5,6,7,8,-Tetrahydropyrido [3,4-d]Pyrimidin4-Amine

### Example 17A

7-Benzyl-N-(4-Trifluoromethylphenyl)-5,6,7,8-Tetrahydropyrido[3,4-d]Pyrimidin-4-Amine

A mixture of ethyl 1-benzyl-3-oxopiperidine-4-carboxylate hydrochloride (6.10 g, 20.5 mmol), formamidine hydrochloride (Aldrich, 1.65 g, 20.5 mmol) and sodium ethoxide (2.7 M in ethanol, 18 mL, 48 mmol) in ethanol (54 mL) was heated to 60° C. and stirred overnight. The mixture was cooled to ambient temperature, concentrated, diluted with water and extracted with dichloromethane. The organic layer was dried (Na₂SO₄), filtered and concentrated. The concentrate was heated in phosphorus oxychloride (Aldrich, 50 mL) at 90° C. for 3 hr. The mixture was cooled to about 25° C., concentrated, diluted with saturated, aqueous NaHCO₃, and extracted with dichloromethane. The organic layer was dried (Na₂SO₄), filtered, concentrated, and purified by flash chromatography, eluted with 25% diethyl ether in hexanes to give the 7-benzyl-4-chloro-5,6,7,8 -tetrahydropyrido[3,4-d]pyrrnidine. To a solution of 7-benzyl4-chloro-5,6,7,8-tetrahydropyrido[3,4-d]pyrimidine (1 g, 4 mmol.) in 10 ml of N,N-dimethylformamide dichlorobis(triphenylphosphine)palladium(II) (100 mg), 4-trifluoromethylaniline (1.2 eq) and sodium tert-butoxide (2 eq) were added. The reaction mixture was heated to 120° C. overnight and cool to room temperature. The reaction was quenched with water and extracted with ethyl acetate (3×50 mL) to give dark brown crude material. The crude material was chromatographed on silica gel, eluting with methanol/dichloromethane to afford the title product.

### Example 17B

N-[4-(Trifluoromethyl)Phenyl]-7-[3-(Trifluoromethyl)Pyridin-2-yl]-5,6,7,8:-Tetrahydropyrido [3,4-d]Pyrimidin-4-Amine

The product of Example 17A was debenzylated using procedure as described in Example 2 to afford N-(4-trifluoromethylphenyl)-5,6,7,8-tetrahydropyrido[3,4-d]pyrimidin-4amine. A mixture of N-(4-trifluoromethylphenyl)-5,6,7,8-tetrahydropyrido[3,4-d]pyrimidin4-amine (217 mg, 0.74 mmol), 2-chloro3-trifluoromethylpyridine (204 mg, 1.1 mmol), and K₂CO₃ (207 mg, 1.5 mmol) in N,N-dimethylformamide (1.5 mL) was microwave-irradiated at 200° C. for 20 min, cooled to about 25° C., diluted with water, and extracted with dichloromethane. The organic extract was dried (Na₂SO₄), filtered and concentrated. The concentrate was chromatographed on silica gel, eluted with 80% diethyl ether in hexane to give the title compound. ¹H NMR (300 MHz, CDCl₃): δ 2.83 (t, J=5.6 Hz, 2 H), 3.72 (t, J=5.8 Hz, 2 H), 4.51 (s, 2 H), 6.54 (s, 1 H), 7.06 (dd, J=7.8, 4.7 Hz, 1 H), 7.63 (d, J=8.5 Hz, 2 H), 7.79 (d, J=8.5 Hz, 2 H), 7.93 (dd, J=7.8, 1.7 Hz, I H), 8.48 (dd, J=4.7, 1.4 Hz, 1 H), 8.65 (s, 1 H). MS (m/z) 440.

5) Biological Activity

In vitro Data—Determination of Inhibition Potencies

Dulbecco's modified Eagle medium (D-MEM)(with 4.5 mg/mL glucose) and fetal bovine serum were obtained from Hyclone Laboratories, Inc. (Logan, Utah). Dulbecco's phosphate-buffered saline (D-PBS)(with 1 mg/mL glucose and 3.6 mg/A Na pyruvate)(without phenol red), L-glutamine, hygromycin B, and Lipofectamine™ were obtained from Life Technologies (Grand Island, N.Y.). G418 sulfate was obtained from Calbiochem-Novabiochem Corp. (San Diego, Calif. ). Capsaicin (8-methyl-N-vanillyl-6-nonenamide) was obtained from Sigma-Aldrich, Co. (St. Louis, Mo.). Fluo4 AM (N-[4-[6-[(acetyloxy)methoxy]-2,7-difluoro-3 -oxo-3H-xanthen-9-yl]-2-[2-[2-[bis[2-[(acetyloxy)methoxy]-2-oxyethyl]amino]-5-methylphenoxy]ethoxy]phenyl]-N-[2-[(acetyloxy)methoxy]-2-oxyethyl]-glycine, (acetyloxy)methyl ester) was purchased from Molecular Probes (Eugene, Or.).

The cDNAs for the human VR1 receptor were isolated by reverse transcriptase-polymerase chain reaction (RT-PCR) from human small intestine poly A+RNA supplied by Clontech (Palo Alto, Calif. ) using primers designed surrounding the initiation and termination codons identical to the published sequences (Hayes et al. Pain 88: 205-215, 2000). The resulting cDNA PCR products were subcloned into pCIneo mammalian expression vector (Promega) and fuilly sequenced using fluorescent dye-terminator reagents (Prism, Perkin-Elmer Applied Biosystems Division) and a Perkin-Elmer Applied Biosystems Model 373 DNA sequencer or Model 310 genetic analyzer. Expression plasmids encoding the hVRl cDNA were transfected individually into 1321N1 human astrocytoma cells using Lipofectamine™. Forty-eight hours after transfection, the neomycin-resistant cells were selected with growth medium containing 800 μg/mL Geneticin (Gibco BRL). Surviving individual colonies were isolated and screened for VR1 receptor activity. Cells expressing recombinant homomeric VRl receptors were maintained at 37° C. in D-MEM containing 4 mM L-glutamine, 300 μg/mL G418 (Cal-biochem) and 10% fetal bovine serum under a humidified 5% CO₂ atmosphere.

The functional activity of compounds at the VR1 receptor was determined with a Ca²⁺influx assay and measurement of intracellular Ca²⁺ levels ([Ca²⁺]i). All compounds were tested over an 11-point half-log concentration range. Compound solutions were prepared in D-PBS (4×final concentration), and diluted serially across 96-well v-bottom tissue culture plates using a Biomek 2000 robotic automation workstation (Beckman-Coulter, Inc., Fullerton, Calif.). A 0.2 μM solution of the VR1 agonist capsaicin was also prepared in D-PBS. The fluorescent Ca²⁺ chelating dye fluo-4 was used as an indicator of the relative levels of [Ca²⁺]i in a 96-well format using a Fluorescence Imaging Plate Reader (FLEPR)(Molecular Devices, Sunnyvale, Calif.). Cells were grown to con fluency in 96-well black-walled tissue culture plates. Then, prior to the assay, the cells were loaded with 100 μL per well of fluo-4 AM (2 μM, in D-PBS) for 1-2 hours at 23 ° C. Washing of the cells was performed to remove extracellular fluo-4 AM (2×1 mL D-PBS per well), and afterward, the cells were placed in the reading chamber of the FLIPR instrument. 50 μL of the compound solutions were added to the cells at the 10 second time mark of the experimental run. Then, after a 3 minute time delay, 50 μL of the capsaicin solution was added at the 190 second time mark (0.05 μM final concentration)(final volume=200 μL) to challenge the VR1 receptor. Time length of the experimental run was 240 seconds. Fluorescence readings were made at 1 to 5 second intervals over the course of the experimental run. The peak increase in relative fluorescence units (minus baseline) was calculated from the 190 second time mark to the end of the experimental run, and expressed as a percentage of the 0.05 μM capsaicin (control) response. Curve-fits of the data were solved using a four-parameter logistic Hill equation in GraphPad Prism® (GraphPad Software, Inc., San Diego, Calif. ), and IC₅₀ values were calculated.

The compounds of the present invention were found to be antagonists of the vanilloid receptor subtype 1 (VR1) receptor with IC₅₀ₛ from about 1 nM to about 10,000 nM. In a preferred range, compounds tested had IC₅₀ₛ from about 1 nM to about 1,000 nM.

In Vivo Data—Determination of Antinociceptive Effect

Experiments were performed on 400 adult male 129J mice (Jackson Laboratories, Bar Harbor, Md.), weighing 20-25 g. Mice were kept in a vivarium, maintained at 22° C., with a 12 hour alternating light-dark cycle with food and water available ad libitum. All experiments were performed during the light cycle. Animals were randomly divided into separate groups of 10 mice each. Each animal was used in one experiment only and was sacrificed immediately following the completion of the experiment. All animal handling and experimental procedures were approved by an IACUC Committee.

The antinociceptive test used was a modification of the abdominal constriction assay described in Collier, et al., Br. J. Pharmacol. Chemother. 32 (1968) 295-310. Each animal received an intraperitoneal (i.p.) injection of 0.3 mL of 0.6% acetic acid in normal saline to evoke writhing. Animals were placed separately under clear cylinders for the observation and quantification of abdominal constriction. Abdominal constriction was defined as a mild constriction and elongation passing caudally along the abdominal wall, accompanied by a slight twisting of the trunk and followed by bilateral extension of the hind limbs. The total number of abdominal constrictions was recorded from 5 to 20 minutes after acetic acid injection. The ED₅₀ₛ were determined based on the i.p. injection.

The other antinociceptive test used was Complete Freund's Adjuvant-induced Thermal Hyperalgesia (CFA) assay described in Pircio et al. Eur J Pharmacol. Vol. 31(2) pages 207-215 (1975). Chronic inflammatory hyperalgesia was induced in one group of rats following the injection of complete Freund's adjuvant (CFA, 50%, 150 μL) into the plantar surface ofthe right hindpaw 48 hours prior to testing. Thermal nociceptive thresholds were measured in three different groups of rats. The ED₅₀ₛ, were determined based on the oral administration. The compound of the present invention tested was found to have antinociceptive effects with ED₅₀ of 22 μmol/kg.

The in vitro and in vivo data demonstrates that compounds of the present invention antagonize the VR1 receptor and are useful for treating pain. Compounds of the present invention are also useful for ameliorating or preventing additional disorders such as, but not limited to, inflammatory thermal hyperalgesia, bladder overactivity, and urinary incontinence as described by Nolano, M. et al., Pain, Vol. 81,pages 135-145, (1999); Caterina, M. J. and Julius, D., Annu. Rev. Neurosci. Vol. 24, pages 487-517 (2001); Caterina, M. J. et al., Science Vol. 288 pages 306-313 (2000); Caterina, M. J. et al., Nature Vol. 389 pages 816-824 (1997); Fowler, C. Urology Vol. 55 pages 60-64 (2000); and Davis, J. et al., Nature Vol. 405 pages 183-187.

The present invention also provides pharmaceutical compositions that comprise compounds of the present invention. The pharmaceutical compositions comprise compounds of the present invention that may be formulated together with one or more non-toxic pharmaceutically acceptable carriers.

The pharmaceutical compositions of this invention can be administered to humans and other mammals orally, rectally, parenterally, intracistemally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrastemal, subcutaneous and intraarticular injection and infusion.

The term “pharmaceutically acceptable carrier,” as used herein, means a non-toxic, inert solid, semi-solid or liquid filler, diluents, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents, which delay absorption such as aluminum monostearate and gelatin.

In some cases, in order to prolong the effect ofthe drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature ofthe particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions, which can be used, include polymeric substances and waxes.

The active compounds can also be in micro-encapsulated form, if appropriate, with one or more ofthe above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 -butylene glycol, dimethyl formamide, oils (in particular, cottonseed, groundnut, corm, germ, olive, castor and sesame oils), glycerol, tetrahydrofurfiryl alcohol, polyethylene glycols and fatty acid esters of sorbitan and mixtures thereof Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof Compositions for rectal or vaginal administration are preferably suppositories which can be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

Compounds ofthe present invention can also be administered in the form of liposomes. As is known in the art, liposomes are generally derived from phospholipids or other lipid substances. Liposomes are formed by mono- or multi-lamellar hydrated liquid crystals, which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art. See, for example, Prescott, Ed., Methods in Cell Biology, Volume XIV, Academic Press, New York, N.Y. (1976), p. 33 et seq.

Dosage forms for topical administration of a compound of this invention include powders, sprays, ointments and inhalants. The active compound may be mixed under sterile conditions with a pharmaceutically acceptable carrier and any needed preservatives, buffers or propellants that may be required. Ophthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated.

When used in the above or other treatments, a therapeutically effective amount of one of the compounds of the present invention can be employed in pure form or, where such forms exist, in pharmaceutically acceptable salt, ester or prodrug form. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder, activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The compounds ofthe present invention can be used in the form of pharmaceutically acceptable salts derived from inorganic or organic acids. The phrase “pharmaceutically acceptable salt”0 means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts are well-known in the art. For example, S. M. Berge et al. describe pharmaceutically acceptable salts in detail in (J. Pharmaceutical Sciences Vol 66, pages 1 et seq, 1977). The salts can be prepared in situ during the final isolation and purification ofthe compounds ofthe invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quatemized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

The term “pharmaceutically acceptable prodrug” or “prodrug,” as used herein, represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I), for example, by hydrolysis in blood.

The present invention contemplates compounds of formula I formed by synthetic means or formed by in vivo biotransformation of a prodrug.

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes ofthe invention.

The total daily dose of the compounds of this invention administered to a human or lower animal may range from about 0.01 to about 100 mg/kg/day. For purposes of oral administration, more preferable doses can be in the range of from about 0.1 to about 25 mg/kg/day. If desired, the effective daily dose can be divided into multiple doses for purposes of administration; consequently, single dose compositions may contain such amounts or submultiples thereof to make up the daily dose.

We claim:

1. A compound having formula (I) or formula (II) 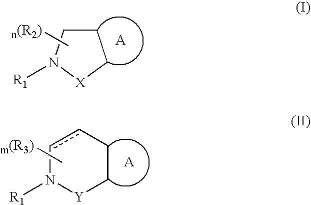
or a pharmaceutically acceptable salt, prodrug, or salt of a prodrug thereof, wherein
X is CH₂ or C(O);
Y is CH₂ or C(O);
R₁ is hydrogen, —C(O)R_{c}, —C(O)N_{c}R_{d}, —S(O)₂R_{c}, aryl, arylalkyl, heteroaryl, heterocycle, cycloalkyl or cycloalkenyl; wherein each aryl, heteroaryl, heterocycle, cycloalkyl, cycloalkenyl or the aryl part of the arylalkyl is independently substituted with 0, 1, 2, 3 or 4 substituents selected from the group consisting of halo, —CN, —NO₂, alkyl, alkenyl, alkynyl, haloalkyl, haloalkoxy, —OR_{d}, —OC(O)R_{d}, —NR_{d}Rₑ, —N(Rₑ)C(O)NRRₑ. —N(Rₑ)C(O)OR_{d}, —N(Rₑ)C(O)NR_{d}Rₑ, —N(Rₑ)S(O)₂R_{d}, —N(Rₑ)S(O)₂NR_{d}Rₑ, —SR_{d}, —S(O)R_{d}, —S(O)₂R_{d}, —S(O)₂NR_{d}Rₑ, —C(O)OR_{d}, —C(O)NR_{d}Rₑ, —alkylOR_{d}, —alkylOC(O)R_{d}, —alkylNR_{d}Rₑ, —alkylN(Rₑ)C(O)OR_{d}, —alkylN(Rₑ)C(O)OR_{d}, —alkylN(Rₑ)C(O)NR_{d}Rₑ, —alkylN(Rₑ)S(O)₂R_{d}, —alkylN(Rₑ)S(O)₂NR_{d}Rₑ, —alkylSR_{d}, —alkylS(O)R_{d}, —alkylS(O)₂R_{d}, —alkylS(O)₂NR_{d}Rₑ, —alkylC(O)OR_{d}, and —alkylC(O)NR_{d}Rₑ;
R₂ is halo, fornyl, —CN, —NO₂, alkyl, alkenyl, alkynyl, haloalkyl, haloalkoxy, —OH, —O(alkyl), —NH_{2,} —NR_{d}Rₑ, —N(alkyl)₂, —N(H)alkyl, —alkylOH, —alkylO(alkyl), —alkylNH_{2,} —alkylN(alkyl)₂, or —alkylN(H)alkyl;
R₃ is halo, forrmyl, —CN, —NO₂, alkyl, alkenyl, alkynyl, haloalkyl, haloalkoxy, —OH, —O(alkyl), —NH_{2,} —N(alkyl)_{2,} —N(H)alkyl, —alkylOH, —alkylO(alkyl), —alkylNH₂, —alkylN(alkyl)₂, or —alkylN(H)alkyl; [symbol]
is a single bond or a double bond;
m is 0, 1, 2 or3;
n is 0, 1 or 2;
A is 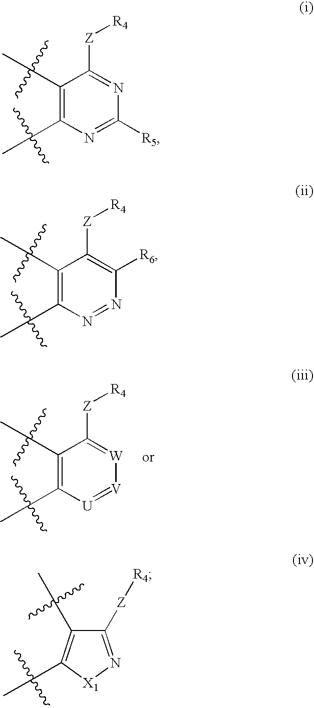
Z is NH, O, or S;
R⁴ is aryl, heteroaryl, heterocycle, cycloalkyl or cycloalkenyl; wherein each R⁴ is substituted with 0, 1, 2, 3 or 4 substituents selected from the group consisting of halo, —CN, —NO₂, alkyl, alkenyl, alkynyl, haloalkyl, haloalkoxy, —OR_{d}, —OC(O)R_{d}, —NR_{d}Rₑ, —N(Rₑ)C(O)NR_{d}Rₑ, —N(Rₑ)C(O)OR_{d}, —N(Rₑ)C(O)NR_{d}Rₑ, —N(Rₑ)S(O)₂R_{d}, —N(Rₑ)S(O)₂NR_{d}Rₑ, —SR_{d}, —S(O)R_{d}, —S(O)₂R_{d}, —S(O)₂NR_{d}Rₑ, —C(O)OR_{d}, —C(O)NR_{d}, heterocycle, —alkylOR_{d}, —alkylOC(O)R_{d}, —alkylNR_{d}Rₑ, —alkylN(Rₑ)C(O)NR_{d}Rₑ, —alkylN(Rₑ)C(O)OR_{d}, —alkylN(Rₑ)C(O)NR_{d}Rₑ, —alkylN(Rₑ)S(O)₂R_{d}, —alkylN(R)S(O)₂NR_{d}Rₑ, —alkylSR_{d}, —alkylS(O)R_{d}, —alkylS(O)₂R_{d}, —alkylS(O)₂NR_{d}Rₑ, —alkylC(O)OR_{d}, and —alkylC(O)NR_{d}Rₑ;
R₅ is hydrogen, halo, haloalkyl, haloalkoxy, —CN, —NO₂, alkyl, —ORₐ, —SRₐ, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};
R₆ is hydrogen, halo, haloalkyl, haloalkoxy, —CN, —NO₂, alkyl, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};
U is CR₇ or N;
V is CR₈ or N;
W is CR₉ or N;
provided that only one of U, V and W is N;
R₇ is H, alkyl, halo, haloalkyl, —CN, —NO₂, —ORₐ, —Se, —NRₐR_{b}, —S(O)Rₐ, —SO₂Rₐ, —alkylNRₐR_{b}, —alkylORₐ, —alkylSRₐ, —alkylS(O)R, —alkylS(O)₂Rₐ, —OC(O)Rₐ, —C(O)ORₐ, —C(O)Rₐ, —C(O)NRₐR_{b}, or R_{c};
R₈ is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or R_{c};
R_{g} is H, alkyl, halo, haloalkyl, —CN, —NO₂, —ORₐ, —SRₐ, —NRₐR_{b}, —S(O)Rₐ, —SO₂Rₐ, —alkylNRₐR_{b}, —alkylORₐ, —alkylSRₐ, —alkylS(O)Rₐ, —alkylS(O)₂Rₐ, —OC(O)Rₐ, —C(O)ORₐ, —C(O)Rₐ, —C(O)NRₐR_{b}, or Rₐ;
X₁ is N(R_{d}),O or S;
Rₐ is hydrogen, alkyl, alkenyl, haloalkyl, R_{f} or —alkylR_{f};
R_{b} is hydrogen, alkyl, alkenyl, haloalkyl, R_{f} or —alkylR_{f};
alternatively, Rₐ and R_{b}, together with the nitrogen atom they are attached to, form a 4, 5 or 6 membered ring selected from the group consisting of heterocycle or heteroaryl, wherein each ring is substituted with 0, 1, 2, 3 or 4 susbstituents selected from the group consisting of oxo, alkyl, —OR_{d}, —NR_{d}Rₐ, —SR_{d}, —S(O)R_{d}, —S(O)₂R_{d}, —alkylOR_{d}, —alkylNR_{d}Rₐ, —alkylSR_{d}, —alkylS(O)R_{d}, —alkylS(O)₂R_{d}, —CN, —NO₂, halo, haloalkyl, and haloalkoxy;
R_{c} is aryl or heteroaryl; wherein each R_{c} is substituted with 0, 1, 2, 3, 4, or 5 substituents selected from the group consisting of alkyl, alkenyl, alkynyl, —OR_{d}, —NR_{d}Rₐ, —SR_{d}, —S(O)R_{d}, —S(O)₂R_{d}, —alkylOR_{d}, —alkyINR_{d}Rₑ, —alkyISR_{d}, —alkylS(O)R_{d}, —alkylS(O)₂R_{d}, —CN, —NO₂, halo, haloalkyl, and haloalkoxy;
R_{d} is hydrogen, alkyl, alkenyl, haloalkyl, R_{f} or —alkylR_{f},
Rₑ is hydrogen, alkyl, alkenyl, haloalkyl, R_{f} or —alkylR_{f}; and
R_{f} is aryl or heteroaryl wherein each Rf is independently substituted with 0, 1, 2, 3, or 4 substituents independently selected from the group consisting of halo, formyl, —CN, —NO₂, alkyl, alkenyl, alyynyl, haloalkyl, haloalkoxy, —OH, —O(alkyl), —NH_{2,} —N(alkyl)_{2,} —N(H)alkyl, —C(O)OH, —C(O)NH₂, —C(O)N(H)(alkyl), —C(O)N(alkyl)₂, —alkylOH, —alkylO(alkyl), —alkylNH_{2,} —alkylN(alkyl)₂, and —alkylN(H)alkyl

2. The compound of formula (I) according to claim 1 wherein
X is CH₂ or C(O);
A is 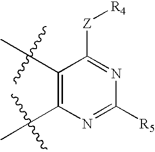
n, R₁, R₂, Z, R₄ and R₅ are as defined in claim 1.

3. The compound according to claim 2 wherein X is CH₂.

4. The compound according to claim 2 wherein X is C(O).

5. The compound of formula (I) according to claim 1 wherein
X is CH₂ or C(O);
A is 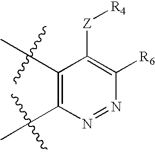
n, R₁, R₂, Z, R₄ and R₆ are as defined in claim 1.

6. The compound according to claim 5 wherein X is CH₂.

7. The compound according to claim 5 wherein X is C(O).

8. The compound of formula (I) according to claim 1 wherein
X is CH₂ or C(O);
A is 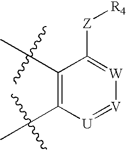
n, R₁, R₂, U, V, W, Z and R₄ are as defined in claim 1

9. The compound according to claim 8 wherein
X is CH₂;
U is N;
V is CR₈;
W is CR₉; and
Z is as defined in claim 1.

10. The compound according to claim 8 wherein
X is CH₂;
U is CR₇;
V is N;
W is CR₉; and
Z is as defined in claim 1.

11. The compound according to claim 8 wherein
X is CH₂;
U is CR₇;
V is CR;
W is N; and
Z is as defined in claim 1.

12. The compound according to claim 8 wherein
X is C(O);
U is N;
V is CR₈;
W is CR₉; and
Z is as defined in claim 1.

13. The compound according to claim 8 wherein
X is C(O);
U is CR₇;
V is N;
W is CR₉; and
Z is as defined in claim 1.

14. The compound according to claim 8 wherein
X is C(O);
U is CR₇;
V is CR₈;
W is N; and
Z is as defined in claim 1.

15. The compound of formula (I) according to claim 1 wherein
X is CH₂ or C(O);
A is 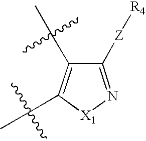
n, R₁, R₂, X₁, Z and R₄ are as defined in claim 1.

16. The compound according to claim 15 wherein
X is CH₂;
Z is NH; and
X₁ is N(R_{d}), O or S.

17. The compound according to claim 15 wherein
X is CH₂;
Z is O; and
X₁ is N(R_{d}), O or S.

18. The compound according to claim 15 wherein
X is CH₂;
Z is NH; and
X₁ is N(R_{d}), O or S.

19. The compound according to claim 15 wherein
X is C(O);
Z is NH; and
X₁ is N(R_{d}), O or S.

20. The compound according to claim 15 wherein
X is C(O);
Z is O; and
X₁ is N(R_{d}), O or S.

21. The compound according to claim 15 wherein
X is C(O);
Z is NH; and
X₁ is N(R_{d}), O or S.

22. The compound of formula (II) according to claim 1, wherein
Y is CH₂ or C(O);
A is 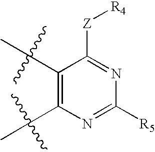
m, R₁, R₃, Z, R₄ and R₅ are as defined in claim 1.

23. The compound according to claim 22 wherein
Y is CH₂;
Z is NH;
m, R₁, R₃, R₄ and R₅ are as defined in claim 1.

24. The compound according to claim 23 wherein R₁ is arylalkyl and R₄ is aryl.

25. The compound according to claim 24 that is
7-benzyl-N-(4-tert-butylphenyl)-5,6,7,8-tetrahydropyrido[3,4-d]pyrimidin-4-amine.

26. The compound according to claim 23 wherein R₁ is heteroaryl and R₄ is aryl.

27. The compound according to claim 26 wherein R₁ is selected from the group consisting of pyridinyl, pyrimidinyl, and thiazolyl.

28. The compound according to claim 27 selected from the group consisting of
N-(4-tert-butylphenyl)-7-(3 -chloropyridin-2-yl)-5,6,7,8-tetrahydropyrido[3,4-d]pyrimidin4-amine,
N-(4 -tert-butylphenyl)-7-pyrimidin-2-yl-5 ,6,7,8 -tetrahydropyrido[3,4-d]pyrimidin-4-amine,
N-(4-tert-butylphenyl)-7-[3 -(trifluoromethyl)pyridin-2-yl]-5,6,7,8-tetrahydropyrido [3,4-d]pyrimidin-4-amine,
2-[4-[(4-tert-butylphenyl)amino]-5,8 -dihydropyrido[3,4-d]pyrimidin-7(6H)-yl]-N,N-dimethylpyridine-3-sulfonamide,
N-(4-tert-butylphenyl)-2-methyl-7-[3-(trifluoromethyl)pyridin-2-yl]-5,6,7,8-tetrahydropyrido [3,4-d]pyrimidin4-amine,
N-(4-tert-butylphenyl)-2-phenyl-7-[3 -(trifluoromethyl)pyridin-2-yl]-5,6,7,8 -tetrahydropyrido [3,4-d]pyrimidin4-amine,
N-(4-tert-butylphenyl)-7-(3 -chloropyridin-2 -yl)-2 -phenyl-5,6,7,8 -tetrahydropyrido [3,4-d]pyrimidin4-amine,
2-tert-butyl-N-(4-tert-butylphenyl)-7-[3 -(trifluoromethyl)pyridin-2-yl]-5,6,7,8 -tetrahydropyrido[3,4-d]pyrimidin-4-amine,
2-tert-butyl-N-(4-tert-butylphenyl)-7-(3 -chloropyridin-2-yl)-5,6,7,8-tetrahydropyrido [3,4-d]pyiimidin-4-amine,
2-tert-butyl-N-(4-tert-butylphenyl)-7-(1,3 -thiazol-2-yl)-5,6,7,8 -tetrahydropyrido[3,4-d]pyrimidin4-amine,
N-(4-tert-butylphenyl)-7-(1,3 -thiazol-2-yl)-5,6,7,8-tetrahydropyiido [3,4-d]pytimidin4-amine,
N-(4 -azepan-1-ylphenyl)-7-pyrimidin-2-yl-5,6,7,8-tetrahydropyrido[3,4 -d]pyiimidin-4-amine,
N-[4-(8-azabicyclo[3.2.1 ]oct-8-yl)-3 -fluorophenyl]-7-[3 -(trifluoromethyl)pyridin-2-yl]-5,6,7,8-tetrahydropyrido[3,4-d]pyrimidin-4-amine,
N-[4 -(8-azabicyclo[3.2.1 ]oct-8 -yl)-3 -fluorophenyl]-7-pyrimidin-2-yl-5,6,7,8 -tetrahydropyrido[3,4-d]pyrimidin4-amine, and
N-[4-(trifluoromethyl)phenyl]-7-[3 -(trifluoromethyl)pyiidin-2-yl]-5,6,7,8-tetrahydropyrido[3,4-d]pyrimidin4-amine.

29. The compound according to claim 23 wherein R₁ is hydrogen and R₄ is aryl.

30. The compound according to claim 29 that is
N-(4-tert-butylphenyl)-5,6,7,8-tetrahydropyrido[3,4-d]pyrimidin-4-amine.

31. The compound according to claim 22 wherein Y is CH₂ and Z is O.

32. The compound according to claim 22 wherein Y is CH₂ and Z is S.

33. The compound according to claim 22 wherein Y is C(O) and Z is NH.

34. The compound according to claim 22 wherein Y is C(O) Z is O.

35. The compound according to claim 22 wherein Y is C(O) Z is S.

36. The compound of formula (II) according to claim 1 wherein
Y is CH₂ or C(O);
A is 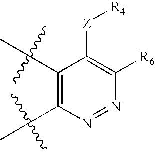
m, R₁, R₃, Z, R₄ and R₅ are as defined in claim 1.

37. The compound according to claim 36 wherein Y is CH₂ and Z is NH.

38. The compound according to claim 36 wherein Y is CH₂ is C and Z is O.

39. The compound according to claim 36 wherein Y is CH₂ and Z is S.

40. The compound according to claim 36 wherein Y is C(O) and Z is NH.

41. The compound according to claim 36 wherein Y is C(O) and Z is O.

42. The compound according to claim 36 wherein Y is C(O) and Z is S.

43. The compound according to formula (II) of claim 1, wherein
Y is CH₂ or C(O);
A is 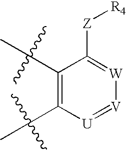
m, R₁, R₃,U, V, W, Z and R₄are as defined in claim 1.

44. The compound according to claim 43 wherein
Y is CH₂;
U is N;
V is CR_{8;} and
W is CR₉.

45. The compound according to claim 43 wherein
Y is CH₂;
U is CR₇;
V is N; and
W is CR₉.

46. The compound according to claim 43 wherein
Y is CH₂;
U is CR₇;
V is CR₈; and
W is N.

47. The compound according to claim 43 wherein
Y is C(O);
U is N;
V is CR₈; and
W is CR₉.

48. The compound according to claim 43 wherein
Y is C(O);
U is CR₇;
V is N; and
W is CR₉.

49. The compound according to claim 43 wherein
Y is C(O);
U is CR₇;
V is CR₈; and
W is N.

50. The compound according to formula (II) of claim 1, wherein
Y is CH₂ or C(O);
A is 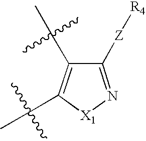
m, R₁, R₃, X₁, Z and R₄ are as defined in claim 1.

51. The compound according to claim 50 wherein
Y is CH₂;
Z is NH; and
X₁ is N(R_{d}), O or S.

52. The compound according to claim 50 wherein
Y is CH₂;
Z is O; and
X₁ is N(R_{d}), O or S.

53. The compound according to claim 50 wherein
Y is CH₂;
Z is NH; and
X₁ is N(R_{d}), O or S.

54. A pharmaceutical composition comprising a therapeutically effective amount of a compound of formula (I) as defined in claim 1 or a pharmaceutically acceptable salt thereof

55. A pharmaceutical composition comprising a therapeutically effective amount of a compound of formula (II) as defined in claim 1 or a pharmaceutically acceptable salt thereof

56. A method of treating a disorder wherein the disorder is ameliorated by inhibiting vanilloid receptor subtype 1 (VR1) receptor in a host mammal in need of such treatment comprising administering a therapeutically effective amount of a compound of formula (I) as defined in claiml or a pharmaceutically acceptable salt thereof, and wherein the disorder is selected form the group consisting of pain, inflammatory hyperalgesia, bladder overactivity and urinary incontinence.

57. A method of treating a disorder wherein the disorder is ameliorated by inhibiting vanilloid receptor subtype 1 (VR 1) receptor in a host mammal in need of such treatment comprising administering a therapeutically effective amount of a compound of formula (II) as defined in claim 1 or a pharmaceutically acceptable salt thereof, and wherein the disorder is selected form the group consisting of pain, inflammatory hyperalgesia, bladder overactivity and urinary incontinence.

58. The method according to claim 56 wherein the disorder is bladder overactivity.

59. The method according to claim 56 wherein the disorder is urinary incontinence.

60. The method according to claim 56 wherein the disorder is pain.

61. The method according to claim 56 wherein the disorder is inflammatory hyperalgesia.

62. The method according to claim 57 wherein the disorder is bladder overactivity.

63. The method according to claim 57 wherein the disorder is urinary incontinence.

64. The method according to claim 57 wherein the disorder is pain.

65. The method according to claim 57 wherein the disorder is inflammatory hyperalgesia.
